(19) United States
(12) Patent Application Publication
(10) Pub. No.: US 2023/0398152 A1
(43) Pub. Date: Dec. 14, 2023

(54) MESENCHYMAL STEM CELLS CO-EXPRESSING CXCR4 AND IL-10 AND USES THEREOF

(71) Applicants: Fundación Instituto de Invesstigación Sanitaria Fundación Jiménez Diaz, Madrid (ES); Centeo de Investigaciones Energéticas, Medioambientales y Tecnológicas, O.A., M.P., Madrid (ES); Consorcio Centro de Investigación Biomédica en Red, Madrid (ES)

(72) Inventors: Rosario Hervas-Salcedo, Madrid (ES); Damian García-olmo, Madrid (ES); Mariano García-Arranz, Madrid (ES); Miriam Hernando Rodríguez, Madrid (ES); Juan Antonio Bueren Roncero, Madrid (ES); Rosa Yáñez González, Madrid (ES); Maria Femández García, Madrid (ES); Mercedes López Santalla, Madrid (ES); Marina Immaculada Garin Ferreira, Madrid (ES)

(21) Appl. No.: 18/025,074

(22) PCT Filed: Sep. 7, 2021
(86) PCT No.: PCT/EP2021/074612

§ 371 Date: Mar. 7, 2023

(30) Foreign Application Priority Data
Sep. 7, 2020 (EP) 20382792.8

(51) Int. Cl. A61K 35/28; C07K 14/54; C07K 14/715; C12N 15/86; A61P 37/06; A61P 29/00; C12N 5/0775

(52) U.S. Cl. CPC A61K 35/28; C07K 14/5428; C07K 14/7158; C12N 15/86; A61P 37/06; A61P 29/00; C12N 5/0667

(57) ABSTRACT

The present invention refers to mesenchymal stem cells (MSCs) characterized in that they are transduced with an integrative expression vector in order to stably co-express the chemokine receptor type 4 CXCR4 and the interleukin EL-10. The present invention also refers to the use of said MSCs as a medicament, particularly in the treatment of inflammatory and/or autoimmune diseases.

### CROSS-REFERENCE TO RELATED APPLICATIONS

This application is a national stage application filed under 35 U.S.C. § 371 of PCT/EP2021/074612, filed Sep. 7, 2021, which claims the benefit of European Patent Application No. EP20382792.8, filed Sep. 7, 2020, all of which is incorporated herein, in their entireties, by reference.

### SEQUENCE LISTING

The Sequence Listing associated with this application is filed in electronic format via EFS-Web and is hereby incorporated by reference into the specification in its entirety. The name of the text file containing the Sequence Listing is 12598170901SEQUENCELISTING.TXT. The size of the text file is 3 KB, and the text file was created on Sep. 7, 2021.

### FIELD OF THE INVENTION

The present invention refers to the medical field. Particularly, the present invention refers to mesenchymal stem cells (MSCs) characterized in that they are transduced with an integrative expression vector in order to stably co-express the chemokine receptor type 4 CXCR4 and the interleukin IL-10. The present invention also refers to the use of said MSCs as a medicament, particularly in the treatment of inflammatory and/or autoimmune diseases.

### PRIOR ART

MSCs are multipotent adult stromal cells with immunomodulatory effects on activated lymphoid cells, including T cells, B cells, natural killer cells, and dendritic cells. MSCs display the ability to home on inflamed sites, where they can modulate inflammatory reactions and contribute to the repair of injured tissues.

In animal models, MSCs have demonstrated their efficacy both in regenerative medicine and also in inflammatory and autoimmune disease models. In phase I/II clinical trials, MSCs have demonstrated a safety profile and showed preliminary evidence of clinical benefit in different diseases such as steroid-resistant graft versus host disease (GVHD), severe systemic lupus erythematosus, complex perianal fistulas, knee osteoarthritis or chronic complete paraplegia, among others. Despite the results obtained in animal models and early-phase clinical trials, only in three Phase III clinical trials the therapeutic efficacy of MSCs has shown statistical significance over standard therapies. These include the treatment of complex perianal fistulas (NCT00475410), steroid-refractory GVHD in children (NCT02336230) and chronic advanced ischemic heart failure (NCT01768702).

Among the parameters that may reduce the therapeutic efficacy of MSCs, it is worth mentioning that the ex vivo expansion of these cells has shown to reduce the modest expression of homing receptors observed in MSCs, and also to induce the senescence in these cells.

Consequently, the present invention is focused on improving the therapeutic efficacy of MSCs, particularly by improving the migration of MSCs towards inflamed sites and also by secreting immunosuppressive and anti-inflammatory cytokines, thus potentiating the therapeutic efficacy of standard unmodified MSCs.

### Description of the Invention

### BRIEF DESCRIPTION OF THE INVENTION

As explained above, the present invention is focused on improving the therapeutic efficacy of MSCs, particularly by enhancing the migration of MSCs towards inflamed sites and by enhancing the release of immunosuppressive and anti-inflammatory cytokines as compared to standard unmodified MSCs.

In order to do so, the inventors of the present invention have used MSCs which have been transduced with an integrative expression vector co-expressing the chemokine receptor type 4 CXCR4 and the interleukin IL-10.

Particularly, a lentiviral vector encoding for CXCR4 and IL-10 was constructed in the context of the present invention. This expression vector was used for transducing MSCs thus co-expressing in a stable manner both CXCR4 and IL-10.

Example 2.1 shows that MSCs transfected with a CXCR4-IL10 mRNA exert anti-inflammatory properties in a mouse model of local inflammation. Nevertheless, these cells do not show enhanced anti-graft versus host disease (GvHD) properties compared to WT MSCs (Example 2.2). In contrast to MSCs transfected with the CXCR4-IL10 mRNA, MSCs that had been transduced with a lentiviral vector carrying the CXCR4-IL10 sequence (Example 2.3), not only exerted enhanced in vitro immunomodulatory properties (Examples 2.4 and 2.5) and local in vivo anti-inflammatory effects compared to WT MSCs (Example 2.3-2.6), but strikingly also developed a significant anti GvHD effect, as shown in Example 2.7 of the present invention.

In fact, the in vitro experiments included in the present invention show that the stable co-expression of these molecules efficiently enhanced the migration of MSCs towards SDF-1 and improved the immunosuppressive properties of these cells. Moreover, the preferential homing of MSCs ectopically expressing CXCR4 and IL10 to inflamed pads was demonstrated in a mouse model in which a local pad inflammation was induced. Taken together, these results demonstrate that the stable co-expression of specific homing and anti-inflammatory molecules, such as CXCR4 and IL10, in human MSCs confers an enhanced anti-inflammatory potential in these cells compared to WT MSCs. The use of this new generation of MSCs transduced with an integrative expression vector co-expressing CXCR4 and IL10 will have a significant impact in clinical cell therapy for the treatment of inflammatory and/or autoimmune diseases.

Consequently, in summary, it is herein proposed the use of MSCs transduced with an integrative expression vector co-expressing both CXCR4 and IL-10 as a medicament, particularly in the treatment of inflammatory and/or autoimmune diseases.

So, the first embodiment of the present invention refers to an expression cassette (hereinafter the expression cassette of the invention) comprising a DNA sequence which in turn comprises: a) a promoter, b) a sequence encoding the chemokine receptor type 4 CXCR4 and c) a sequence encoding interleukin IL-10. In a preferred embodiment, the expression cassette further comprises a regulatory element for increasing transgene expression. In a preferred embodiment, the regulatory element is the woodchuck hepatitis virus regulatory element (WPRE) RNA export signal sequence or a functional variant or fragment thereof. In a preferred embodiment, the expression cassette further comprises, between the sequence encoding the chemokine receptor type 4 CXCR4 and the sequence encoding interleukin IL-10, a sequence which encodes an autocatalytic peptide. In a preferred embodiment, the autocatalytic peptide is E2A. In a preferred embodiment the promoter is a human phosphoglycerate kinase (PGK) promoter sequence or a functional homolog or variant thereof. In a preferred embodiment, the expression cassette comprises in the order 5 ‘ to 3’: a) a human phosphoglycerate kinase (PGK) promoter sequence or a functional homolog or variant thereof, b) a sequence encoding the chemokine receptor type 4 CXCR4, c) a sequence encoding the autocatalytic peptide E2A, d) a sequence encoding interleukin IL-10; and d) the woodchuck hepatitis virus post-transcriptional regulatory element (WPRE).

In a preferred embodiment, the expression cassette comprises non-native codon optimized sequences of the human genes CXCR4 (SEQ ID NO: 1) and IL10 (SEQ ID NO: 3). In a preferred embodiment, the sequence coding the autocatalytic peptide E2A is SEQ ID NO: 2, which is used to ease the co-expression of both molecules (CXCR4 and IL10).

The second embodiment of the present invention refers to a recombinant gene delivery vector (hereinafter the recombinant gene delivery vector of the invention) comprising the above defined expression cassette. In a preferred embodiment, the recombinant gene delivery vector is a lentiviral vector. In a preferred embodiment, the vector of the invention is an integrative vector which is permanently incorporated into the host chromosomes.

The third embodiment of the present invention refers to a cell (hereinafter the cell of the invention) comprising the expression cassette or the recombinant gene delivery vector of the invention. In a preferred embodiment, the cells are MSCs derived from bone marrow, placenta, umbilical cord, amniotic membrane, menstrual blood, peripheral blood, salivary gland, skin and foreskin, synovial fluid, amniotic fluid, endometrium, adipose tissue, cord blood and/or dental tissue.

The fourth embodiment of the present invention refers to a pharmaceutical composition comprising the recombinant gene delivery vector or the cell of the invention and, optionally, pharmaceutically acceptable excipients or carriers.

The fifth embodiment of the present invention refers to the gene delivery vector or the cells of the invention for use as a medicament. In a preferred embodiment, the present invention refers to the gene delivery vector or the cells of the invention for use in the treatment of inflammatory diseases and/or autoimmune diseases, for instance Graft-versus-host disease (GvHD), sepsis or rheumatoid arthritis. Alternatively, this embodiment refers to a method for treating inflammatory diseases and/or autoimmune diseases, for instance Graft-versus-host disease (GvHD), sepsis or rheumatoid arthritis, which comprises the administration to the patient of a therapeutically effective dose or amount of the gene delivery vector or the cells of the invention, or a pharmaceutical composition comprising thereof.

For the purpose of the present invention the following terms are defined:
By “comprising” it is meant including, but not limited to, whatever follows the word “comprising”. Thus, use of the term “comprising” indicates that the listed elements are required or mandatory, but that other elements are optional and may or may not be present.
By “consisting of” it is meant “including, and limited to”, whatever follows the phrase “consisting of”. Thus, the phrase “consisting of” indicates that the listed elements are required or mandatory, and that no other elements may be present.
“Pharmaceutically acceptable excipient or carrier” refers to an excipient that may optionally be included in the pharmaceutical composition of the invention and that causes no significant adverse toxicological effects to the patient.
By “therapeutically effective dose or amount” the present invention refers to the situation when the cells or the pharmaceutical composition are administered as described above and brings about a positive therapeutic response in a subject having an inflammatory or autoimmune disease. The exact amount required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

### BRIEF DESCRIPTION OF THE FIGURES

FIG. 1. Evidence of in vivo efficacy of MSCs transfected with the bicistronic CXCR4-IL10 mRNA in a mouse model of local inflammation. Enhanced anti-inflammatory effect of MSCs transfected with the CXCR4-IL10 mRNA is observed as compared to WT-MSCs.

FIG. 2. Absence of in vivo efficacy of MSCs transfected with the bicistronic CXCR4-IL10 mRNA in a mouse model of GVHD. A) Survival curve B) Weight and C) Clinical score.

FIG. 3. (A) Design of the DNA bicistronic lentiviral vector used to co-express CXCR4 and IL-10. (B) Levels of CXCR4, (C) IL-10 secretion and (D) vector copy number per cell (VCN/Cell) in CXCR4/IL10-MSCs compared to WT-MSCs. N.D. Not detectable

FIG. 4. In vitro characterization of MSCs transduced with the DNA PGK-CXCR4-IL10 lentiviral vector. A) Immunophenotype of CXCR4/IL10-MSCs compared to WT-MSCs. B) Differentiation capacity of CXCR4/IL10-MSCs to bone tissue compared to WT-MSCs. C) Differentiation capacity of CXCR4/IL10-MSCs to adipose tissue compared to WT-MSCs.

FIG. 5. Enhanced migration capacity of MSCs transduced with the DNA PGK-CXCR4-IL10 lentiviral vector compared to WT-MSCs. A) Representative picture of the migration ability of WT-MSCs and CXCR4/IL10-MSCs in response to SDF-1. B) Quantification of the migration ability of WT-MSCs and CXCR4/IL10-MSCs in response to SDF-1.

FIG. 6. Enhanced in vitro immunosuppression capacity of MSCs transduced with the DNA PG-CXCR4-IL10 lentiviral vector. A) Scheme of the experimental system used to evaluate the in vitro immunosuppressive activity of MSCs. B) CXCR4/IL10-MSCs showed improved immunosuppression capacity than WT-MSCs.

FIG. 7. Enhanced in vivo efficacy of MSCs transduced with the DNA PGK-CXCR4-IL10 lentiviral vector in a mouse model of local inflammation. A) Scheme of the experimental system used to evaluate the in vivo anti-inflammatory effect of WT-MSCs and CXCR4/IL10-MSCs. B) Enhanced anti-inflammatory effect of MSCs transduced with the PGK-CXCR4-IL10 lentiviral vector compared to WT-MSCs.

FIG. 8. Enhanced anti-GvHD of MSCs transduced with the DNA PGK-CXCR4-IL10 LV compared to WT-MSCs: Analysis of the GvHD clinical signs. A) Scheme of the experimental system used to evaluate the in vivo anti-GVHD of WT-MSCs and CXCR4/IL10-MSCs. B) GVHD score comparing different experimental groups.

FIG. 9. Enhanced anti-GvHD of MSCs transduced with the DNA PGK-CXCR4-IL10 LV compared to WT-MSCs: A) Flow cytometry analysis of human CD45+ cells in peripheral blood of recipient mice showing a reduced expansion of xenogenic donor leukocyte in the GVHD humanized mouse model. B) Flow cytometry analysis of human CD45⁺ cells in spleen in a GVHD humanized mouse model confirming the reduced infiltration of xenogenic donor leukocyte in this immune organ.

FIG. 10. Enhanced anti-GvHD of MSCs transduced with the DNA PGK-CXCR4-IL10 LV compared to WT-MSCs: Analysis of the infiltration of donor lymphocytes expressing IFN-g or IL10: A) Reduced content of INFg-secreting human T cells responsible for GVHD disease in the spleen of NSG mice that had been infused with CXCR4-IL10-MSCs. B) Increased content of IL10-secreting human T cells in the spleen of NSG mice with GVHD treated with CXCR4-IL10-MSCs.

FIG. 11. Enhanced anti-GvHD of MSCs transduced with the DNA PGK-CXCR4-IL10 LV compared to WT-MSCs: Quantification of human factors in recipient mice by qPCR. A) Analysis of pro-inflammatory factors (IFNg, IL-17 and IL-22) in the spleen of NSG treated with WT-MSC or CXCR4/IL10-MSCs. B) Analysis of anti-inflammatory factors (IL-5 or FoxP3) in the spleen of NSG treated with WT-MSC or CXCR4/IL10-MSCs.

FIG. 12. Evolution of weight and GVHD clinical score in NSG mice transplanted with human mononuclear cells and infused with WT or CXCR4/IL10-MSCs. (A) Evolution of the weight shown as a percentage over time, assuming that the weight of day 0 corresponds to 100%. (B) Clinical score of the disease determined over time in the different transplanted groups. The overall GVHD score was evaluated in terms of weight loss, posture, activity, hair texture, skin integrity, and presence of diarrhea. * p<0.05, ** p<0.01, *** p<0.001

FIG. 13. Flow cytometric analysis of circulating human cells in peripheral blood three weeks after transplantation in NSG mice transplanted with human mononuclear cells and infused with WT or CXCR4/IL10-MSCs. (A) Percentage of circulating human CD45⁺ cells. (B) Percentage of circulating human CD3⁺ T cells. (C) Characterization of CD3⁺ T cells as human CD4⁺, CD8⁺ or CD4⁺CD8⁺T cells. Each bar represents the mean±SEM. * p<0.05, ** p<0.01, *** p<0.001.

FIG. 14. Phenotypic characterization of circulating human CD4⁺ and CD8⁺T cells (naïve, effector and memory T cel) in NSG mice transplanted with human mononuclear cells and infused with WT or CXCR4/IL10-MSCs. (A) Effector T/naïve T cell ratio in the CD4⁺T cells. (B) Effector T/naïve T cell ratio in the CD8⁺T cells. Each bar represents the mean±SEM. * p<0.05, ** p<0.01.

FIG. 15. Analysis by flow cytometry of activation markers in circulating human T cells in peripheral blood three weeks after NSG mice transplantation with human mononuclear cells and infused with WT or CXCR4/1L10-MSCs. Activation profile of T cells labeled as CD3⁺CD45±. Each bar represents the mean±SEM of data from two different experiments with MNCs from two different donors (n=10-12 mice/group). * p<0.05.

FIG. 16. Analysis of exhaustion markers in circulating human CD3⁺T cells in peripheral blood three weeks after of NSG mice with human mononuclear cells and infused with WT or CXCR4/1L10-MSCs. Inhibition profile of CD3⁺CD45+ human T cells

FIG. 17. Human cytokines and growth factor levels involved in GVHD in the serum of NSG transplanted mice three weeks after transplantation with human mononuclear cells and infused with WT or CXCR4/1L10-MSCs. Each bar represents the mean±SEM. * p<0.05, ** p<0.01, *** p<0.001

FIG. 18. Analysis by flow cytometry of human hematopoietic cells in the spleen three weeks after transplantation of NSG mice with human mononuclear cells and infused with WT or CXCR4/1L10-MSCs. Percentage of circulating human CD45⁺ cells distributed as CD3⁺, CD19±, CD56±, CD14⁺ and CD15⁺ cells. Each bar represents the mean±SEM. * p<0.05, ** p<0.01, *** p<0.001

FIG. 19. Phenotypic characterization of T cell subpopulations in the spleen. (A) Distribution of human CD4⁺, CD8⁺ or double positive T cells within the population of CD3⁺CD45⁺ cells. (B) Distribution of naïve, effector and memory subpopulations in the CD4⁺T cell population. (C) Distribution of naïve, effector and memory subpopulations in the CD8⁺T cell population. Each bar represents the mean±SEM

FIG. 20. Analysis by flow cytometry of activation profile in human T cells in spleen three weeks after NSG mice transplantation with human mononuclear cells and infused with WT or CXCR4/1L10-MSCs. (A) Activation profile of CD3⁺CD45+ labeled T cells. (B) Activation profile of the CD4⁺T cell subpopulation. (C) Activation profile of the CD8⁺T cell subpopulation. Each bar represents the mean±SEM. * p<0.05, ** p<0.01

FIG. 21. Analysis by flow cytometry of exhaution profile in human T cells in spleen three weeks after transplantation of NSG mice with human mononuclear cells and infused with WT or CXCR4/1L10-MSCs. (A) Inhibition profile of CD3⁺CD45⁺T cells. (B) Inhibition profile of the CD4⁺T cell subpopulation. (C) Activation profile of the CD8⁺T cell subpopulation. Each bar represents the mean±SEM. * p<0.05.

FIG. 22. Phenotypic characterization of human CD19⁺B cell subpopulations in spleen (naive B cells CD24-CD38-CD27⁻; transitional B cells CD24^{low/+}CD38⁺CD27⁻; memory B cells and plasma cells CD24^{low/+}CD38⁺CD27±). Each bar represents the mean±SEM. * p<0.05.

FIG. 23. Flow cytometry analysis of the human B cell polarization towards regulatory B cells in the spleen of NSG mice three weeks after transplantation with human mononuclear cells and infused with WT or CXCR4/1L10-MSCs. (A) Representative flow cytometric analysis of each group and graphical representation of IL10⁺ transitional B cell percentage. (B) Representative flow cytometric analysis of each group and graphical representation of the IL10⁺ memory B cell percentage. Each bar represents the mean±SEM. * p<0.05.

FIG. 24. Histopathological analysis in the lungs of NSG mice transplanted with human mononuclear cells and infused with WT or CXCR4/1L10-MSCs. (A) Representative images of H/E staining (left), human anti-CD3 immunohistochemical staining (center), and human anti-CD8 immunohistochemical staining (right). (B) Quantification of infiltating CD3⁺T cells in the lungs. (C) Quantification of infiltating CD8⁺ T cells in the lungs. Each bar represents the mean±SEM. * p<0.05, ** p<0.01,*** p<0.001, **** p<0.0001.

FIG. 25. Histopathological analysis in the liver of NSG mice transplanted with human mononuclear cells and infused with WT or CXCR4/1L10-MSCs. (A) Representative images of H/E staining (left), human anti-CD3 immunohistochemical staining (center), and human anti-CD8 immunohistochemical staining (right). (B) Quantification of infiltrating CD3⁺T cells in the liver. (C) Quantification of infiltrating CD8⁺ T cells in the liver. Each bar represents the mean±SEM. * p<0.05, ** p<0.01,*** p<0.001, **** p<0.0001.

FIG. 26. Experimental design on DSS-induced colitis. Different concentrations of dextran sulphate sodium (DSS) were used with ranges from 2.5% to 3% in drinking water during 7 days ad libitum. A single dose of WT or CXCR4/IL10-MSCs (3×10⁶ cells/mouse) was intraperitoneally infused at day 5. For long-term evaluation, a re-challenge with 7-day cycle of DSS in drinking water was performed 12 weeks later.

FIG. 27. DSS-induced colitic status of mice following intraperitoneal administration WT-MSCs or CXCR4/1L10-MSCs. Disease activity index (DAI) (A), fold-change in body weight (B) and survival (C). Representative images of colon tissue (magnification 4× and 10×) at day 10 following the treatment with DSS (D). Healthy, n=14; DSS, n=26; DSS+WT-MSCs, n=21 and DSS+CXCR4/IL10-MSCs, n=26 Data are presented by mean and standard error of the disease activity index and of the fold-change in body weights, with respect to Day 0 expressed by percentage over time. Survival are presented by percentage. Significance was analyzed by the Mann-Whitney U test and long rank test and represented by *p<0.05 and ****p<0.0001 DSS vs Healthy; $ p<0.05 DSS+WT-MSCs vs DSS; & p<0.05 and && DSS CXCR4/IL10-MSCs vs DSS and #p<0.05 DSS CXCR4/IL10-MSCs vs DSS WT-MSCs. Results correspond to 5 independent experiments.

FIG. 28. DSS-induced colitic status of mice after the third month of administration of WT-MSCs or CXCR4/1L10-MSCs. Disease activity index (DAI) (A), fold-change in body weight (B) and survival (C). Healthy, n=10; DSS, n=15; DSS+WT-MSCs, n=15 and DSS+sCXCR4-IL10-MSCs, n=15 Data are presented by mean and standard error of the disease activity index and of the fold-change in body weights, with respect to Day 0 expressed by percentage over time. Survival are presented by percentage. Significance was analyzed by the Mann-Whitney U test and long rank test and represented by **p<0.01 and ****p<0.0001 DSS vs Healthy, & p<0.05 DSS+CXCR4/IL10-MSCs vs DSS and #p<0.05 and ##p<0.01 DSS+CXCR4/IL10-MSCs vs DSS+WT-MSCs. Results correspond to 3 independent experiments

### DETAILED DESCRIPTION OF THE INVENTION

The present invention is illustrated by means of the examples set below without the intention of limiting its scope of protection.

### Example 1. Material and Methods

### Example 1.1. Generation and Expansion of Adipose-Derived MSCs (Ad-MSCs)

Adipose tissue samples were obtained by surgical resection from healthy donors after informed consent. Adipose tissue was disaggregated and digested with collagenase A (Serva, Germany) at a final concentration of 2 mg/ml for 4 hours at 37° C. Digested samples were filtered through 100 μm nylon filters (BD Bioscience, USA) and centrifuged for 10 minutes. The cell pellet was re-suspended in α-MEM (Gibco, USA) supplemented with 5% platelet lysate (Cook medical, USA), 1% penicillin/streptomycin (Gibco) and 1 ng/ml human basic fibroblast growth factor (bFGF, Peprotech, USA). Cells were seeded at a concentration of 10,000 cells/cm2 in culture flasks (Corning, USA) and cultured at 37° C. For the expansion of Ad-MSCs, cell medium was changed every 2-4 days and adherent cells were serially passaged using 0.25% trypsin/EDTA (Sigma-Aldrich, USA) upon reaching near confluence (70%-90%). For in vitro and in vivo studies, Ad-MSCs were used at passages from 4 to 8.

### Example 1.2. Characterization of WT-MSCs and CXCR4/IL10-MSCs

WT-MSCs and MSC that had been transduced with the CXCR4-IL10 lentiviral vector (CXCR4/IL10-MSCs) were immunophenotypically characterized by flow cytometry (Fortessa, BD Bioscience, USA) as described by the Mesenchymal cell kit (Immunostep, Spain). The monoclonal anti-human antibodies included in these studies were the following: CD29, CD44, CD73, CD90, CD105, CD166, CD45, CD19, HLA-DR, CD14 and CD34. Data were analysed with FlowJo version X (FlowJo LLC, USA).

The osteogenic and adipogenic differentiation ability of Ad-MSCs was determined using the NH-OsteoDiff and NH-AdipoDiff Media (Miltenyi Biotec, Germany), respectively, according to manufacturer's protocols. Alcaline phosphatase deposits were seen after the staining with Fast BCIP/NCP (Sigma-Aldrich) while lipid droplets were seen with optic microscopy (Nikon, Germany).

### Example 1.3. Construction of the DNA CXCR4/IL10 Lentiviral Vector

The fragment containing the lentiviral backbone and the PGK promoter (7362 bp) was obtained by simultaneous digestion of pCCL.PGK.FANCA.Wpre*plasmid (9087 bp) with Agel and SacII restriction enzymes (New England Biolabs, USA), whose restriction sites were blanking FANCA transgene at 5′ and 3′-end, respectively.

Digested lentiviral backbone without transgene was purified from agarose gel with NucleoSpin Gel and PCR Clean-up kit (Macherey-Nagel, Germany). Fragments containing codon-optimized sequences of human CXCR4 and IL10 were obtained by polymerase chain reaction (PCR) taking pUC57 plasmids used for mRNA synthesis as a template. PCR of each plasmid was performed using two specific primers which included Agel and SacII restriction sites at 5′-end and the first or last 20 bp of the CXCR4 or IL10 transgenes Amplification was carried out following Herculase II Fusion Enzyme's protocol (Agilent, USA) depending on target size, without using dimethyl sulfoxide (DMSO) and stablishing 58° C. for annealing temperature. PCR products were simultaneous digested with Agel and SacII and also purified by column using NucleoSpin Gel and PCR Clean-up kit.

Digested lentiviral backbone and fragments of interest were ligated with the T4 DNA Ligase (New England Biolabs) maintaining target:vector ratio at 5:1. Ligated products were transformed into Stable3 bacteria to obtain pCCL.PKG-CXCR4-IL10.Wpre*plasmid.

### Example 1.4. Lentiviral Vector Production

All self-inactivating HIV-1-derived vectors used in this work were produced by a second-generation packaging system in HEK293T cells, obtaining VSV-G-pseudotyped viruses. A total amount of 12×106 cells were plated the day before in 150 mm diameter plates. Transfections were performed on cells at 70-80% confluence in 150 mm diameter plates following the CaCl₂) DNA precipitation methods previously described. Briefly, one hour before transfection culture medium was replaced by fresh DMEM-Glutamax containing 10% HyClone (GE Healthcare, USA) and 1% penicillin/streptomycin. Equimolecular mixtures of three plasmids containing the transgenes, the viral genome and the packaging constructs were prepared freshly. HEK293T cells of each plate were transfected with 22,5 μg of the gene transfer plasmid, 12 μg of the pMD2.VSVg envelope plasmid (PlasmidFactory, Germany) carrying the heterologous VSVg envelope and 27,5 μg of the pCMVdR8.74 packaging plasmid (PlasmidFactory) carrying the gag-pol-rev viral genes. These plasmid mixtures were prepared in a final volume of 3,8 ml of ultra-pure H₂O and 450 μl of 2.5M CaCl2 were carefully added. After a 5 min incubation at room temperature, 3,8 ml of 2×Hank's Buffered Saline (HBS) buffer (100 mM HEPES (Gibco), 281 mM NaCl, 1.5 mM Na2HPO4, pH=7.13) was added drop by drop, allowing the formation of Ca2+ precipitates. This solution was added to HEK293T cells that would integrate those precipitates. Five hours after, medium containing precipitates was replaced by fresh medium. Supernatants were collected at 48 hours post-transfection. They were harvested, filtered using a 0.22 μm pore-size filter (Millipore, Merck KGaA, Germany) and concentrated by ultra-centrifuging at 20,000 rpm and 4° C. for 2 hours. Then, viral pellets were suspended in DMEM for at least 1 h at 4° C., spun down to discard cellular debris and stored at −80° C. in aliquot of 100 μl.

### Example 1.5. Transduction of the Ad-MSCs

Two different strategies were carried out to transduce human Ad-MSCs: transduction of adhered MSCs and transduction of MSCs in suspension. In this set of experiments, transduction enhancers (TEs) were added during the transduction process with the aim of increasing the transduction efficacy.

### Example 1.6. CXCR4 and IL-10 Protein Co-Expression

The expression of CXCR4 on the cell surface of Ad-MSCs was determined by flow cytometry after labelling with a PE-conjugated anti-human CXCR4 antibody for 30 min at 4° C. (Biolegend, USA). IL10 levels secreted by Ad-MSCs were measured in the supernatant of cultured cells using the human IL10 Quantikine ELISA Kit (R&D System, USA). Total protein extracts were isolated from Ad-MSCs using the RIPA buffer (ThermoFisher Scientific, USA) containing a protease inhibitor mixture (Merck Millipore, Germany) Twenty micrograms of each of the cell lysates were resolved in 4-12% polyacrylamide gels (Bio-Rad, USA) and transferred to PVDF membranes (Bio-Rad). Membranes were blocked with 5% v/v nonfat dry milk in 0.1% Tween-20 PBS. Samples were immunoblotted by incubation with rabbit monoclonal anti-human CXCR4 antibody (Abcam, UK) diluted in blocking solution. Mouse anti-human Vinculin (Abcam) was used as a loading control. Blots were visualized with Clarity Western ECL substrate (Bio-Rad) using a ChemiDoc MP System and ImageLab sofware (Bio-Rad).

### Example 1.7. Cell Migration Assay

Migration assays were carried out in transwells with an 8 μm pore polycarbonate membrane insert (Costar, Cambridge, MA). 5×103 Ad-MSCs were placed in the upper insert chamber of the transwell assembly. The lower chamber contained murine or human SDF-1 (Peprotech, USA) at a final concentration of 100 ng/ml. Twenty-four hours after incubation, the upper part of the membrane was scrapped gently by a cotton swab to remove non-migrating cells and washed with PBS. The membrane was fixed with 3.7-4% formalin overnight at 4° C. and stained with haematoxylin for 4 hours at RT. The number of migrating cells was determined by the scoring of four random fields per well under the Nikon Eclipse E400 microscope (10×) (Nikon, UK) and pictures were obtained with a Leica DFC420 camera (Leica, UK).

### Example 1.8. In Vitro Immunosuppression Assay

Peripheral blood mononuclear cells (MNCs) were obtained by Ficoll-Paque PLUS (GE Healthcare Bioscience, Sweden) density gradient from heparinized peripheral blood samples obtained from healthy donors after informed consent. Before co-culture, MNCs were marked with the intracellular fluorescent dye CFSE (carboxyfluorescein diacetate succinimidyl ester, Molecular Probe, USA), following a previously described protocols. WT-MSCs and CXCR4/IL10-MSCs were plated in 24-well plates at a concentration of cells/well. Twenty-four hours later, 5×105 MNCs were added to each well in presence of 10 μg/mL of phytohemagglutinin (PHA) (Sigma-Aldrich) to induce the T cell proliferation. After 3 days of incubation, cells harvested from culture wells were analysed by flow cytometry for cell proliferation. Data were analysed with ModFit LT™ (Verity Software House, USA).

### Example 1.9. Quantification of secreted cytokines and factors

WT-MSCs and CXCR4/IL10-MSCs were seeded in 6-well plates at a concentration of 1×105 cells/well. At 4 h post-transfection, supernatants were collected and secreted PGE2 and TGF131 were quantified by ELISA (R&D System, USA). Secreted IL-6, IFNγ and TNFα were quantified by flow cytometry using LEGENDplex™ Human Th Cytokine Panel (Biolegend, USA) following manufacturer's protocol.

### Example 1.10. Gene expression analysis

RNA from WT-MSCs and CXCR4/IL10-MSCs was isolated using RNAeasy® Plus Mini Kit and reverse transcribed with RETROscript (ThermoFisher Scientific, Waltham, USA). cDNA was subjected to quantitative Real-Time PCR (qPCR) using FastStart Universal SYBR Green Master master mix (Roche, Indianapolis, USA) and specific primers for human interleukins and different factors. qPCRs were run on a 7,500 fast real-time PCR system (ThermoFisher Scientific). Results were normalized to human GAPDH expression and expression of control samples according to the 2^{-ΔΔCt} method.

### Example 1.11. LPS-Induced Inflamed Pad Model

FVB/NJ mice were housed in the animal facility (Registration No. ES280790000183) at CIEMAT (Madrid, Spain). Mice were routinely screened for pathogens in accordance with FELASA procedures and received water and food ad libitum. All experimental procedures were carried out according to Spanish and European regulations (Spanish RD 53/2013 and Law 6/2013, European Directive 2010/63/UE). Procedures were approved by the CIEMAT Animal Experimentation Ethical Committee according to approved biosafety and bioethics guidelines. FVB/NJ mice were sedated and administered a single injection of 40 μg of E. coli LPS in 30 μl of PBS into the right pad. Similarly, 30 μL of PBS were injected into the left pad, as a control. Four hours after Ad-MSCs transfection, 5×105 WT-MSCs or CXCR4/IL10-MSCs were intravenously infused through the tail vein. Pad inflammation was determined by measuring the thickness with a digital calliper at 24, 48 and 72 h following LPS administration. At the end of the experiments, mice were sacrificed by CO 2 inhalation.

Peripheral blood cells were collected to analyse the mouse haematological parameters using the hematology analyzer Abacus (Diatron, USA).

### Example 1.12. Humanized Mouse Model of Graft-Versus-Host Disease (GvHD) in NSG Mice

To establish the model, NSG mice were irradiated with 2Gy and the following day they were transplanted with 5×10⁶ human MNCs. Three days later, one million of WT-MSCs or CXCR4/IL10-MSCs were infused intravenously Animals were weighed daily and monitored for possible symptoms of GVHD such as weight loss, hunched back, ruffling of hair and diarrhea. The severity of GVHD was graded from 0 (absence of GVHD) to 8 (severe GVHD). Animals were sacrificed humanely when they exhibited the euthanasia GVHD criteria (>20% weight loss or a score≥6.5).

### Example 1.13. Statistical Analysis

Statistical analyses were performed using Graph Pad Prism 7.0 software (Graph Pad Software, USA). Data of in vitro tests are expressed as mean±standard deviation (SD) and as mean±the standard error of the mean (SEM) in in vivo tests. Normal distribution was analyzed by the Shapiro-Wilks test. To compare more than two groups, parametric test (one-way ANOVA) for normal distribution and non-parametric test (Kruskal-Wallis test) for non-normal distribution were used. Appropriate post hoc analysis to compare means was performed. P values<0.05 were considered statistically significant.

### Example 1.14. Histopathological Analysis in a GVHD Mouse Model

Lungs and livers were surgically removed and fixed with formalin overnight. After fixation, the tissue samples were processed in a standard way, embedding them in paraffin for the generation of a block. To assess tissue morphology, 3-5 μm sections of the paraffin blocks were made with a microtome and hematoxylin-eosin staining was performed using standard techniques. The interpretation of the tissues following previously established GVHD grading systems.

### Example 1.15. Immunohistochemical Analysis in a GVHD Mouse Model

The slides with the samples were deparaffinized and rehydrated following standard protocols. Lung and liver samples were labeled with human CD3 and CD8. Antigen unmasking of CD3-labeled samples was carried out using a sodium citrate buffer (1.8 mM citric acid monohydrate and 8.2 mM trisodium citrate dihydrate; pH 6) using a pressure cooker (Dako, Agilent Technologies). For the unmasking of the samples stained with CD8, a Tris-EDTA buffer (Target Retrieval Solution pH 9; Dako) and the same pressure cooker were used. Endogenous peroxidase was inhibited with 0.2% hydrogen peroxide dissolved in methanol for 10 minutes. Nonspecific epitopes were blocked with 10% horse serum dissolved in PBS for 30 minutes at 37° C. The primary antibodies were incubated overnight at 4° C. diluted in the blocking solution. The secondary antibodies, conjugated with biotin, were incubated for one hour at room temperature diluted in the blocking solution. To amplify the signal, a biotin-avidin-peroxidase system (VECTASTAIN elite ABC HRP kit, Vector Laboratories) was used, incubating for 30 minutes at room temperature. The signal was visualized using diaminobenzidine as the peroxidase substrate (DAB Kit, Vector Laboratories). Finally, the samples were counterstained with hematoxylin, dehydrated using standard procedures, and mounted using a mounting adhesive (CV Mount, Leica Biosystems). Images were taken with an optical microscope (Olympus BX41) and a digital camera (Olympus DP21). The analysis of the percentage of marking in each of the samples was carried out with the ImageJ program.

### Example 1.16. Induction and Evaluation of Dextran Sulphate Sodium (DSS)-Induced Colitis

Different concentrations of dextran sulphate sodium (DSS; 36,000-50,000 MW, MI′ Biomedicals, Irvine CA USA) were used with ranges from 2.5% to 3% in drinking water for 7 days ad libitum. A single dose of native or CXCR4/IL-10-modified MSCs (3×10⁶ cells/mouse) was intraperitoneally infused at day 5.

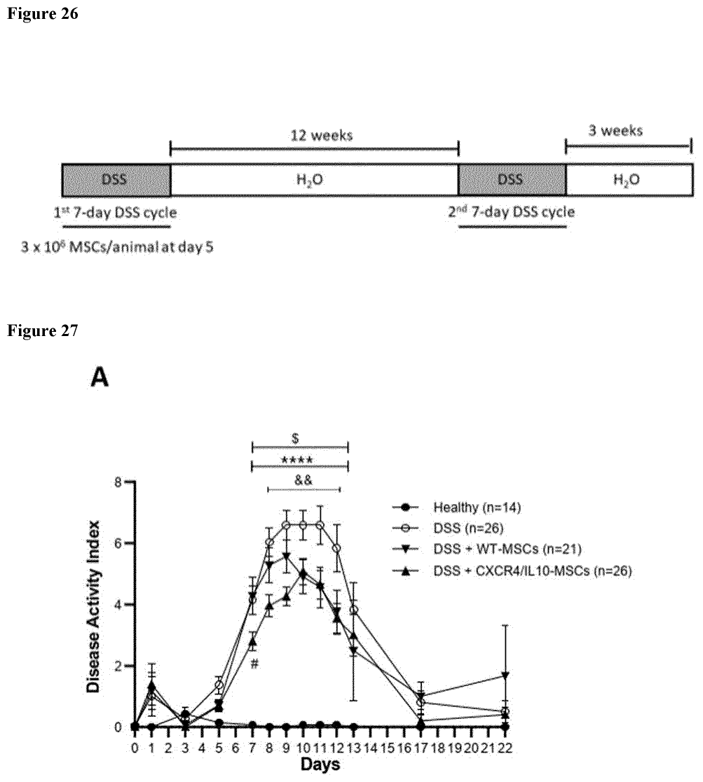

For long-term evaluation, a re-challenge with 7-day cycle of DSS in drinking water was performed (FIG. 26).

Colitis score or disease activity index (DAI) was defined as follows: (1) Body weight loss (0: no loss; 1: 1%-5%; 2: 5%-10%; 3: 10%-20%, 4: >20% loss of weight and 5: no survival); (2) stool consistency (0: normal stools; loose stools; 2: watery diarrhoea; 3: watery diarrhoea with blood and 4: no survival) and (3) the general physical activity (0: normal; 1-2: moderate activity; 3: null activity and 4: no survival). The fold-change in body weight was calculated by the difference in body weight at a defined time-point with respect to the initial body weight at day 0 just before the beginning of DSS treatment expresses as percentage.

Colitis score was also evaluated by colon histological analysis. Colons were surgically removed and fixed with formalin overnight. At 48 h, 1-cm colon tissues were cut and embedded in paraffin and stained with haematoxylirdeosin. The sections were examined for infiltrating mononuclear cells and analysis of the intestinal epithelial and submucosa structures using an optical microscope.

### Example 2. Results

For the sake of clarity, kindly note that the results provided in Examples 2.1 and 2.2, with respect to MSCs transfected with bicistronic CXCR4-IL10 mRNA, are just included as comparative examples to show how these results were improved when the MSCs were transduced with an integrative expression vector co-expressing CXCR4 and IL10 (Examples 2.3 to 2.7).

### Example 2.1. MSCs Transfected with the Bicistronic CXCR4-IL10 mRNA Exhibit Significant Local Anti-Inflammatory Effects

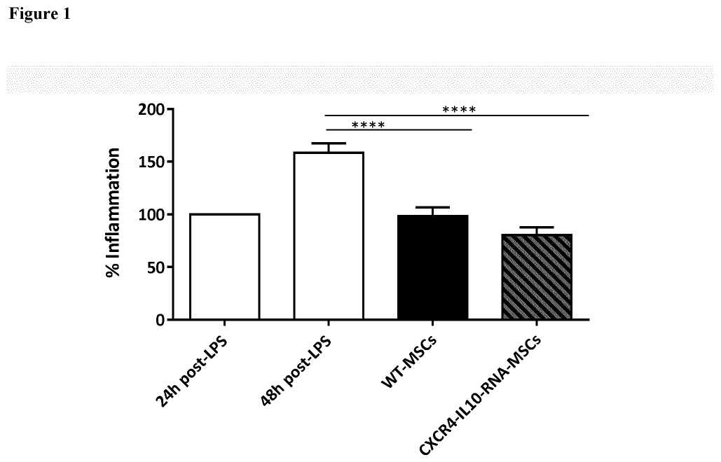

We tested the in vivo efficacy of MSCs transfected with the bicistronic CXCR4-IL10 mRNA in a mouse model of local inflammation model induced by LPS. Both WT and CXCR4-IL10-RNA-MSCs were able to exhibit significant anti-inflammatory effects, although MSCs transfected with the bicistronic CXCR4-IL10 mRNA were significantly more efficient compared to WT-MSCs. See FIG. 1 wherein it is shown the analysis of the in vivo efficacy of MSCs transfected with the bicistronic CXCR4-IL10 mRNA in a mouse model of local inflammation. Enhanced anti-inflammatory effect of MSCs transfected with the CXCR4-IL10 mRNA is observed as compared to WT-MSCs.

### Example 2.2. Absence of In Vivo Efficacy of MSCs Transfected with the Bicistronic CXCR4-IL10 mRNA in a Graft Versus Host Disease Mouse Model

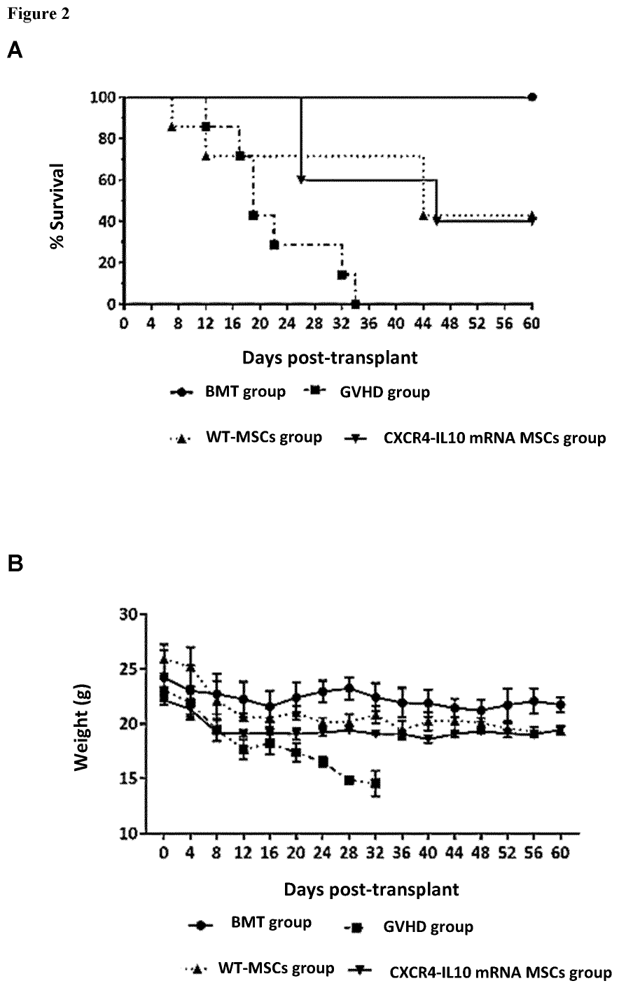

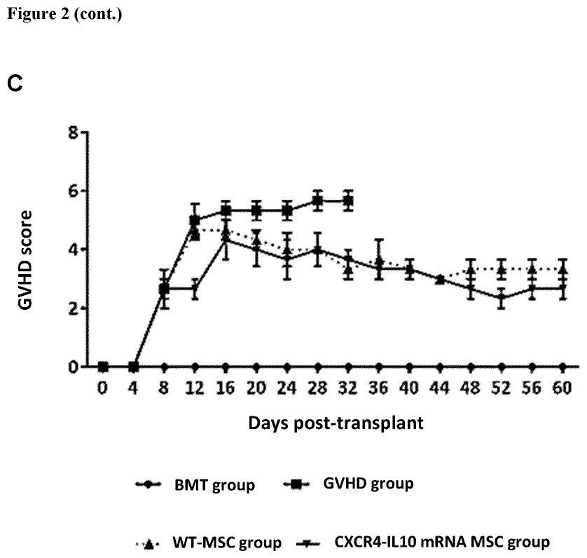

We also tested the in vivo efficacy of MSCs transfected with the bicistronic CXCR4-IL10 mRNA in a graft versus host disease mouse model. A mouse model of haploidentical hematopoietic transplantation was conducted by transplanting BM cells from C57B1/6 donor mice into B6D2F1 recipient mice, previously irradiated with a lethal dose of 11 Gy. All recipients were injected intravenously with 10×10⁶ BM donor cells. To induce graft versus host disease (GVHD), recipients also received a total number of 2×10⁸ donor splenocytes. One day after GVHD induction, mice were administered saline (GVHD group), WT-MSCs or mRNA-transfected MSCs (1×10⁶) via the tail vein. Transplanted recipients were observed daily for symptoms of GVHD such as weight loss, hunched back, ruffling of hair and diarrhea. The severity of GVHD was graded from 0 (absence of GVHD) to 8 (severe GVHD). Animals were sacrificed humanely when they exhibited the euthanasia GVHD criteria (>20% weight loss or a score≥6.5). FIG. 2 shows the analysis of the in vivo efficacy of MSCs transfected with the bicistronic CXCR4-IL10 mRNA in a mouse model of GVHD. A) Survival curve B) Weight and C) Clinical score. As shown in FIG. 2A, we did not observe any difference between the WT-MSCs and CXCR4-IL10 mRNA MSCs to inhibit GVHD.

### Example 2.3. Generation of MSCs Transduced with a Bicistronic DNA CRCR4-IL10 Lentiviral Vector for Improving the Efficacy of WT MSCs to Inhibit Graft Versus Host Disease

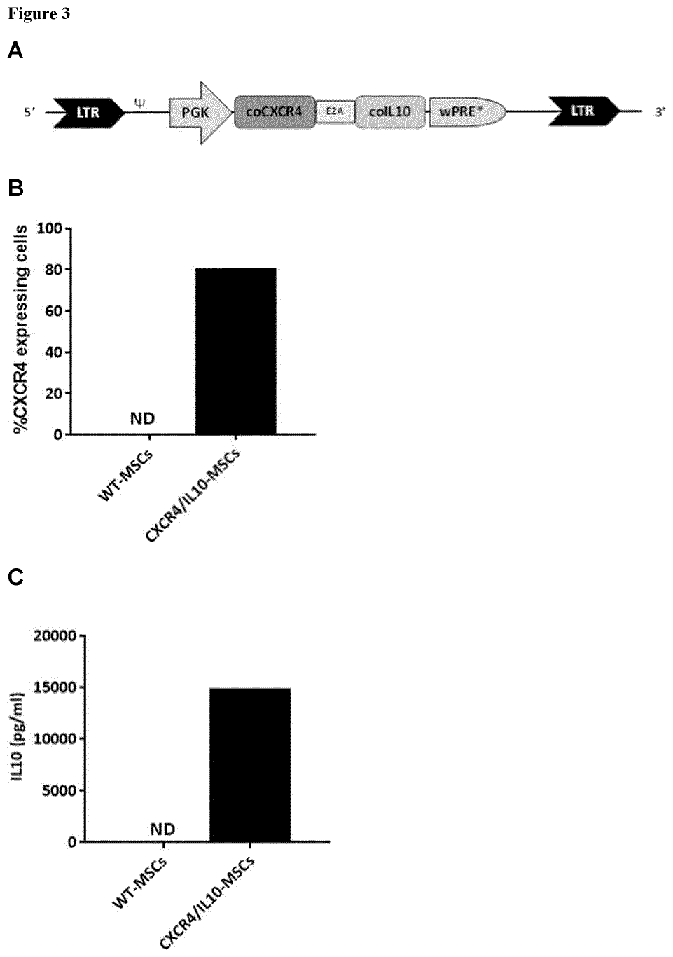

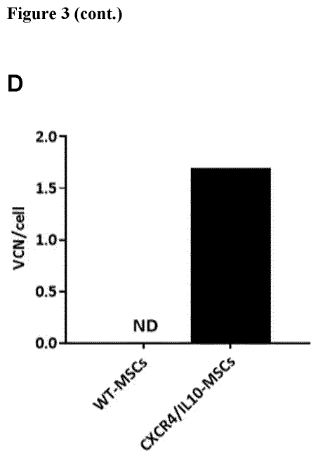

In these studies, we have generated a lentiviral vector in which the optimized sequences of the CXCR4 and IL10 genes have been cloned in a bicistronic lentiviral vector under the human physiological promoter PGK (FIG. 3A).

After testing different methods of Ad-MSCs transduction as well as different amounts of the vector, a population of modified Ad-MSCs (CXCR4/IL10-MSCs) was obtained. This population of CXCR4/IL10-MSCs overexpressed CXCR4, around 80% MSCs was positive to CXCR4. Higher concentrations of IL10 were secreted by CXCR4/IL10-MSCs compared to unmodified MSCs (WT-MSCs). The vector copy number was analyzed in these CXCR4/IL10-MSCs by qPCR (FIG. 3B).

### Example 2.4. In Vitro Characterization of CXCR4/1L10-MSCs Compared to WT-MSC

MSCs modified with the bicistronic PGK-CXCR4-IL10 lentiviral vector were characterized following the criteria established by the ISCT (International Society of Cellular Therapy) for mesenchymal cells.

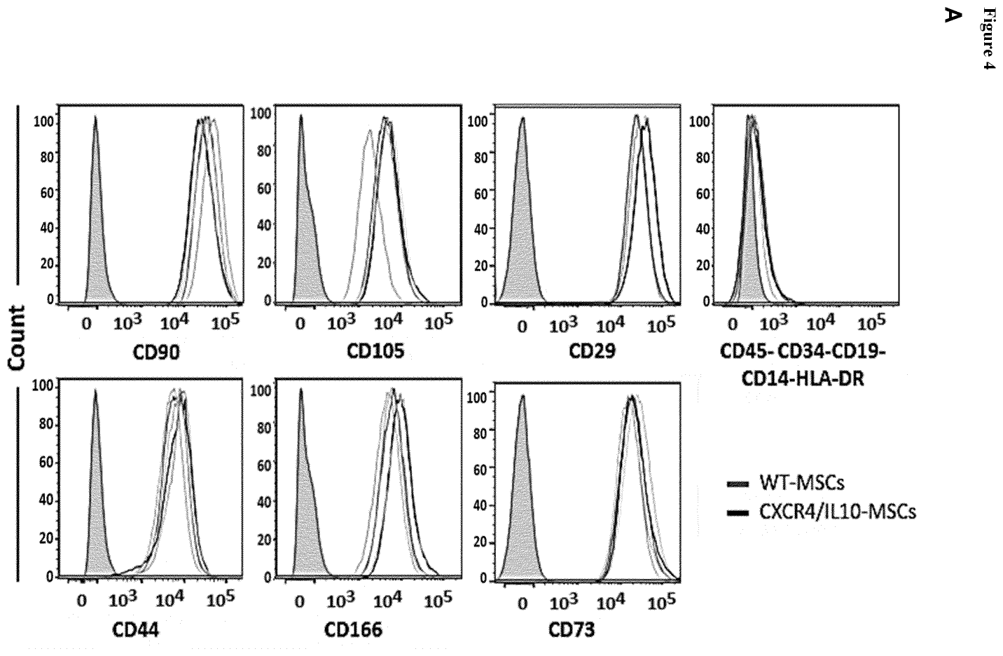

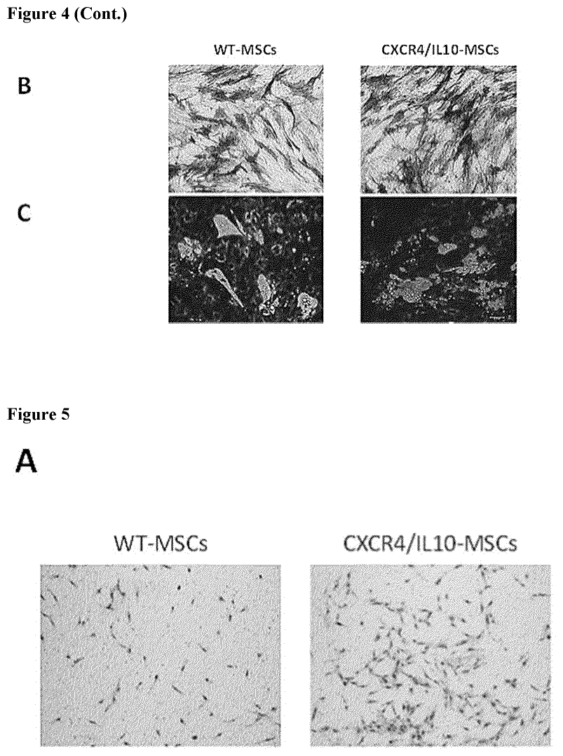

The in vitro characterization showed that the modification of the MSCs with the bicistronic lentiviral vector did not affect their immunophenotype (FIG. 4A) nor their ability to differentiate to bone (FIG. 4B) and adipose (FIG. 4C) tissue compared to unmodified mesenchymal cells (WT-MSCs).

### Example 2.5. In Vitro Functionality of CXCR4/1L10-MSCs Compared to WT-MSC

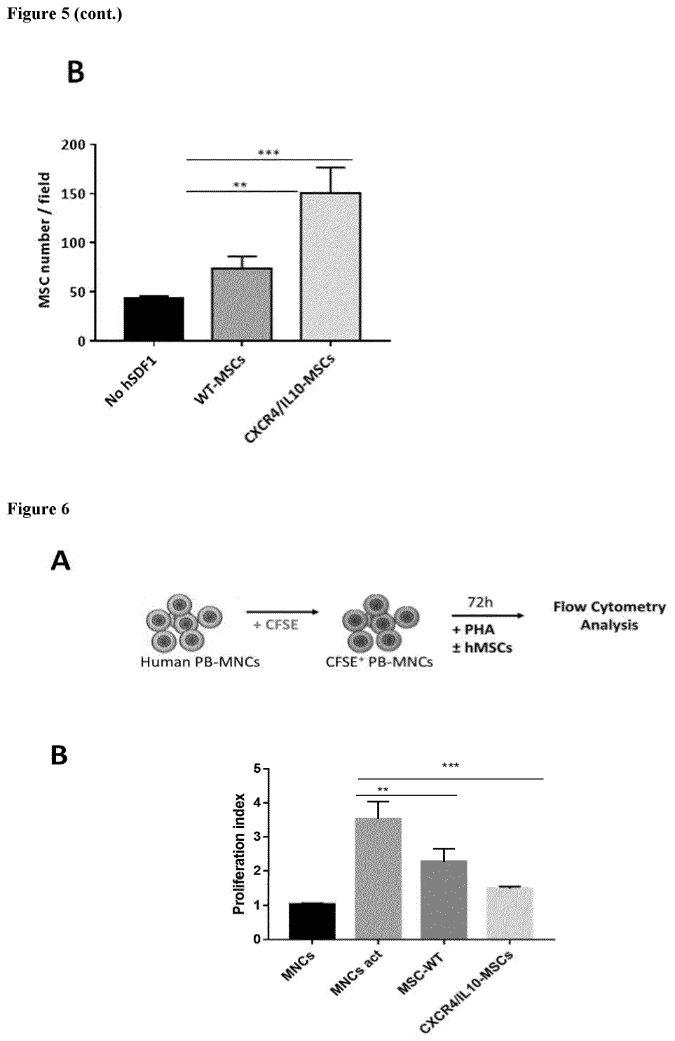

To study the in vitro functionality of mesenchymal cells modified with the bicistronic lentiviral vector (CXCR4/IL10-MSCs), a transwell migration assay was first performed in response to SDF-1, ligand of CXCR4 (FIG. 5A). The results of this assay showed an enhanced migration ability of CXCR4/IL10-MSCs as compared to WT-MSCs (FIG. 5B).

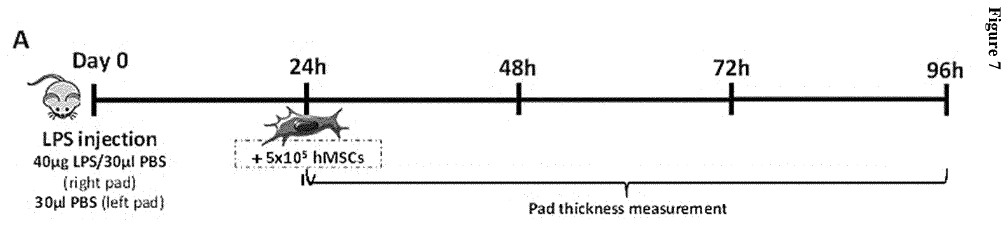

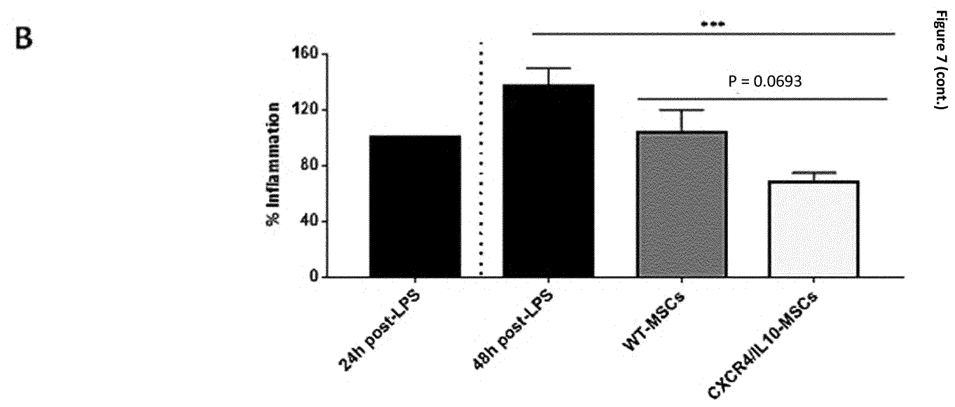

The second in vitro functional characterization study consisted of an immunosuppression assay in which the ability of the CXCR4/IL10-MSCs to inhibit the proliferation of activated mononuclear cells (MNCs) was evaluated compared to WT-MSCs (FIG. 6A).

As already described, WT-MSCs showed a high capacity to inhibit the proliferation of activated MNCs. However, this inhibition was significantly higher when MSCs were transduced with the PGK-CXCR4-IL10 lentiviral vector (FIG. 6B). These studies demonstrate that the transduction of MSCs with the bicistronic lentiviral vector significantly improve the immunomodulatory capacity of these cells compared to WT-MSCs.

### Example 2.6. Enhanced In Vivo Efficacy of CXCR4/IL10-MSCs to Inhibit Local Inflammation Compared to WT-MSC

To test the in vivo efficacy of MSCs transduced with the PGK-CXCR4-IL10 lentiviral vector, cells were tested in a mouse model of local inflammation model induced by LPS.

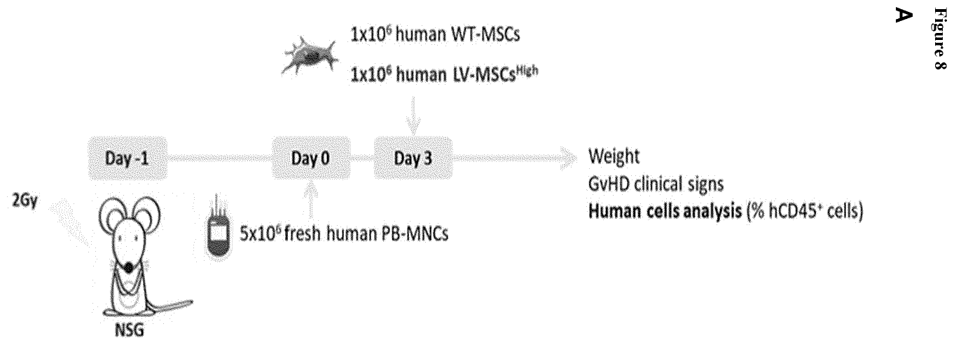

The LPS was injected on the right pad of each mouse. One day after LPS injection, the different types of Ad-MSCs (WT-MSCs and CXCR4/IL10-MSCs) were infused intravenously (n=7−14 mice/group). Inflammation was measured macroscopically with a digital caliper, using the left pad as a control in each mouse (FIG. 7A).

The results showed that 24 hours after the infusion of the Ad-MSCs (48 hours after the LPS injection), all the mice that had received Ad-MSCs controlled the inflammation, while the inflammation continued to grow in the group of mice that had only received the LPS injection.

However, the control of the inflammation was statistically higher in the group of mice that had received CXCR4/IL10-MSCs (FIG. 7B).

### Example 2.7. Improved Efficacy of MSCs Transduced with the DNA Bicistronic Lentiviral Vector to Inhibit Graft-Versus-Host Disease (GvHD) Compared to WT MSCs

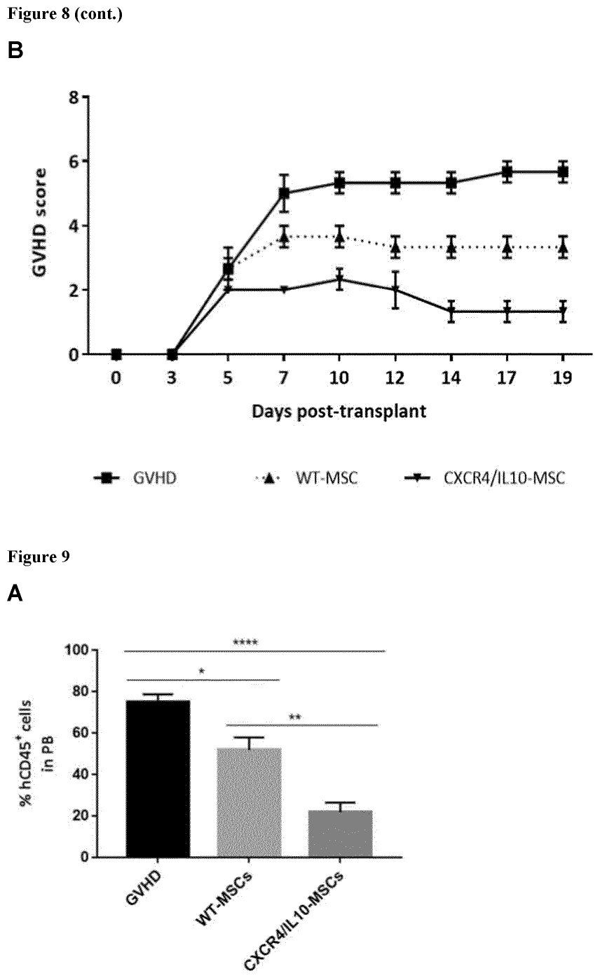

The therapeutic efficacy of MSCs transduced with the bicistronic lentiviral vector was also tested in a graft-versus-host disease (GvHD) mouse model based on the infusion of peripheral blood human mononuclear cells (MNC) in immunodeficient NSG mice (FIG. 8A). To establish the model, mice were irradiated with 2Gy and the following day they were transplanted with 5×10⁶ human MNCs. Three days later, one million of WT-MSCs or CXCR4/IL10-MSCs were infused intravenously. Animals were weighed daily and monitored for possible key signs of GVHD (FIG. 8B).

As FIG. 8B shows, GVHD score was significant better in the group of NSG mice that received CXCR4/IL10-MSCs, comparing not only with GVHD groups but also WT-MSCs group.

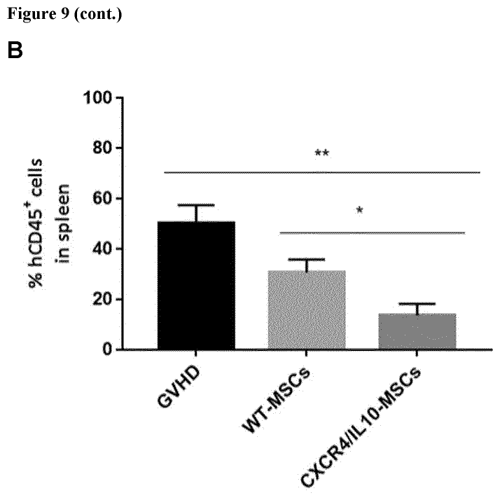

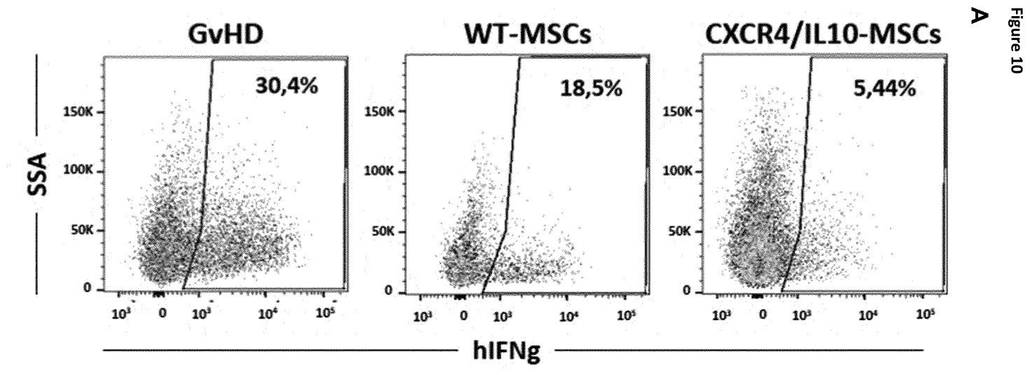

Two weeks after the infusion of MNCs, mice that only received human MNCs (GvHD group) began to show signs of the disease (weight loss, hunched back). Therefore, at this time recipient mice from all the three groups were sacrificed to analyze the percentage of human CD45⁺ cells in the peripheral blood (PB) and in the spleen (SP). It was found that the percentage of infiltrating human CD45⁺ cells was significantly reduced in mice that received WT-MSCs. Nevertheless, the reduction observed both in PB and spleen was significantly higher in mice that were infused with CXCR4/IL10-MSCs (FIG. 9A-B).

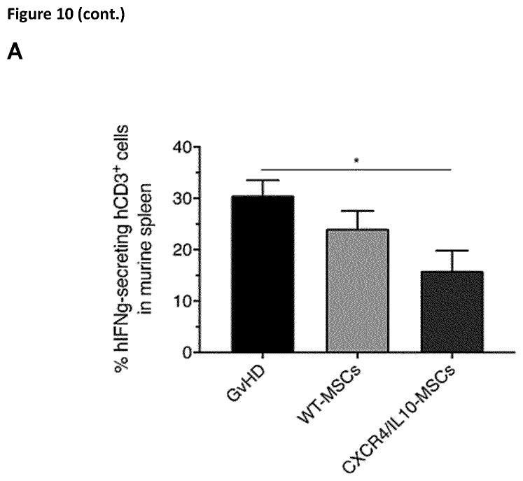

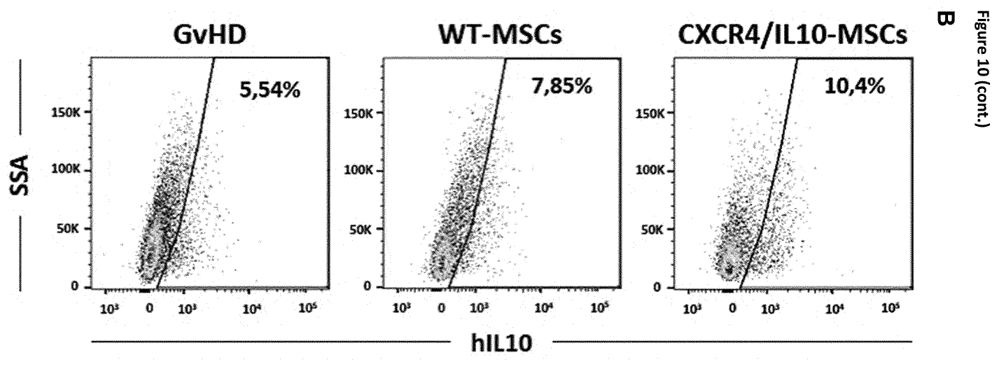

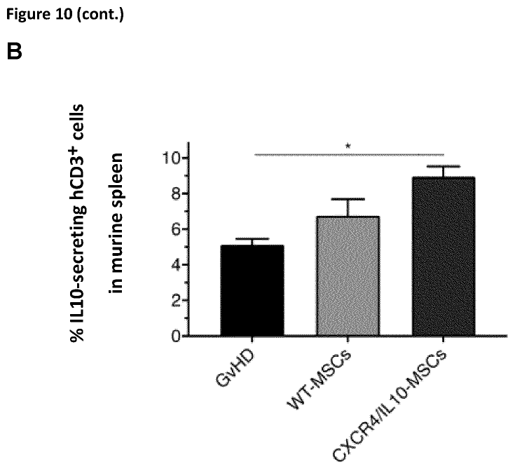

Human CD45±CD3⁺ cells responsible for GVHD disease were analyzed by flow cytometry in the GVHD humanized mouse model. Remarkably, NSG mice treated with CXCR4/IL10-MSCs, but not with WT-MSCs, showed a statistically reduced percentage of pro-inflammatory T cells (CD3⁺IFNg⁺) compared to the GvHD control group (FIG. 10A). In addition, a statistically significant increase in the percentage of anti-inflammatory T cells (CD3⁺IL10±) was also observed (FIG. 10B) in the group that received CXCR4/IL10-MSCs, but not with WT-MSCs, compared to the GvHD control group.

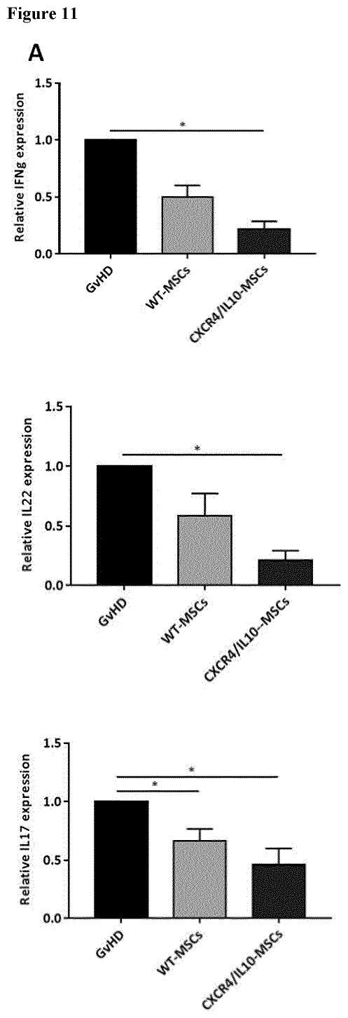

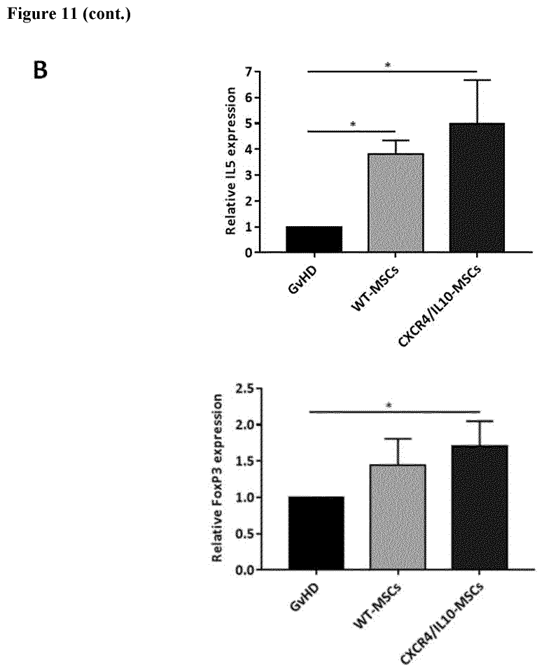

These data observed by flow cytometry were confirmed by qPCR. Pro-inflammatory factors such as IFNg, IL-17 and IL-22 were significantly reduced in the case of mice that received CXCR4/IL10-MSCs, but not WT-MSCs, compared to the control GvHD group. Quantification of the levels of expression of anti-inflammatory factors such as IL-5 or FoxP3 showed that these factors were statistically increased in the case of mice that received CXCR4/IL10-MSCs, but not WT-MSCs, with respect to the control GvHD group (FIG. 11).

### Example 2.8. In Vivo Efficacy of CXCR4/IL10-MSCs Tested in a Humanized Model of Graft Versus Host Disease (GvHD)

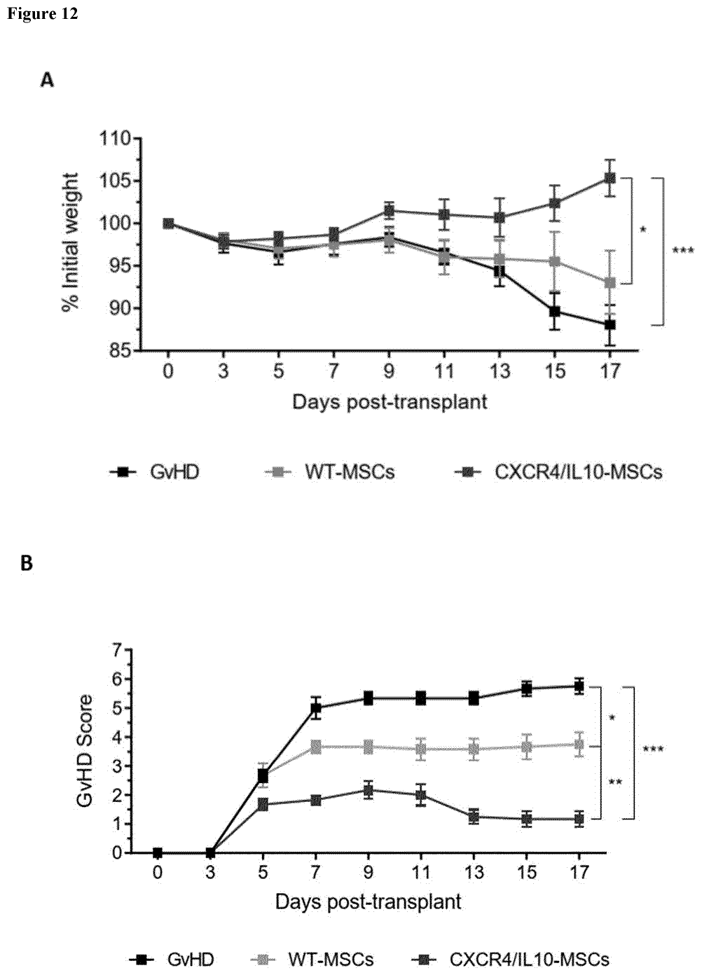

To test the in vivo efficacy of CXCR4/IL10-MSCs with respect to WT-MSCs, a humanized model of graft versus host disease (GvHD) was developed. The greatest weight loss was observed in the GvHD group that did not receive any type of Ad-MSCs. Furthermore, compared to the remarkable weight loss observed in the GvHD group and also in the group that received WT-MSCs, no weight loss was observed in the group that received CXCR4/IL10-MSCs (FIG. 12A). Moreover, although the group that received WT-MSCs presented a lower GvHD score (milder clinical signs) compared to non-MSC-treated mice, the GvHD score was significantly lower in mice that received CXCR4/IL10-MSCs (FIG. 12B).

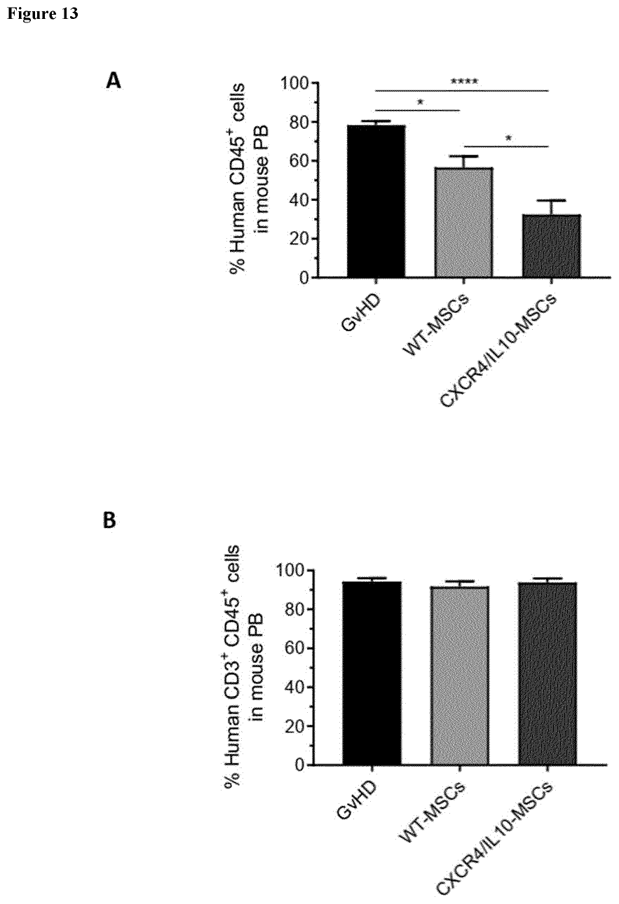

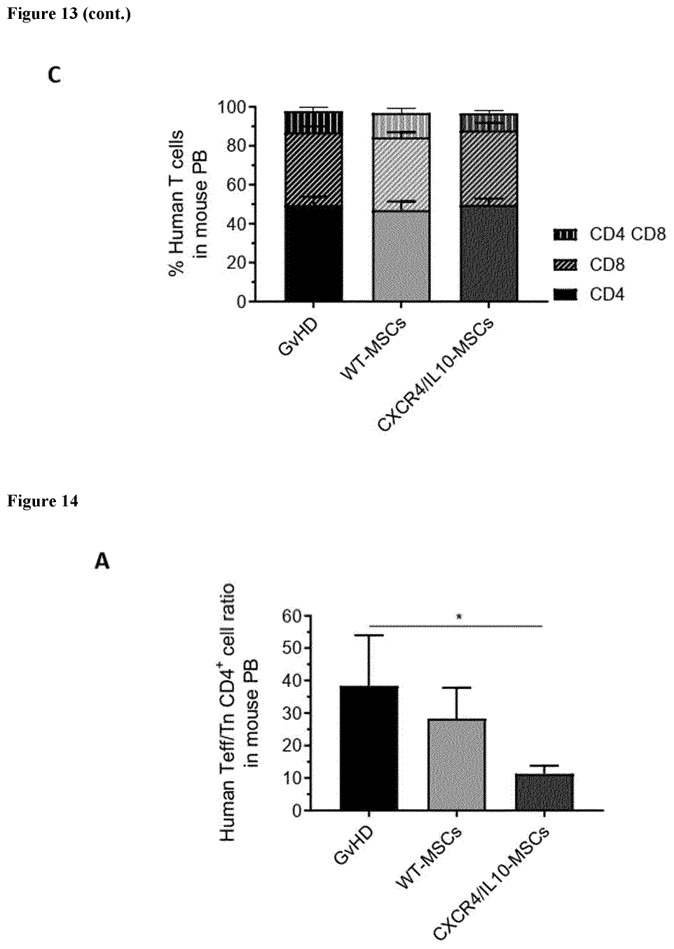

The analysis of human leukocytes in the peripheral blood of transplanted mice showed significant reductions in mice that received Ad-MSCs (% hCD45 cells; FIG. 13A). Nevertheless, mice treated with CXCR4/IL10-MSCs showed the lowest proportion of human leukocytes, most of which were human CD3⁺T cells in all instances (FIG. 13B), and with no differences among CD4⁺, CD8⁺ or double positive T cells (FIG. 13C).

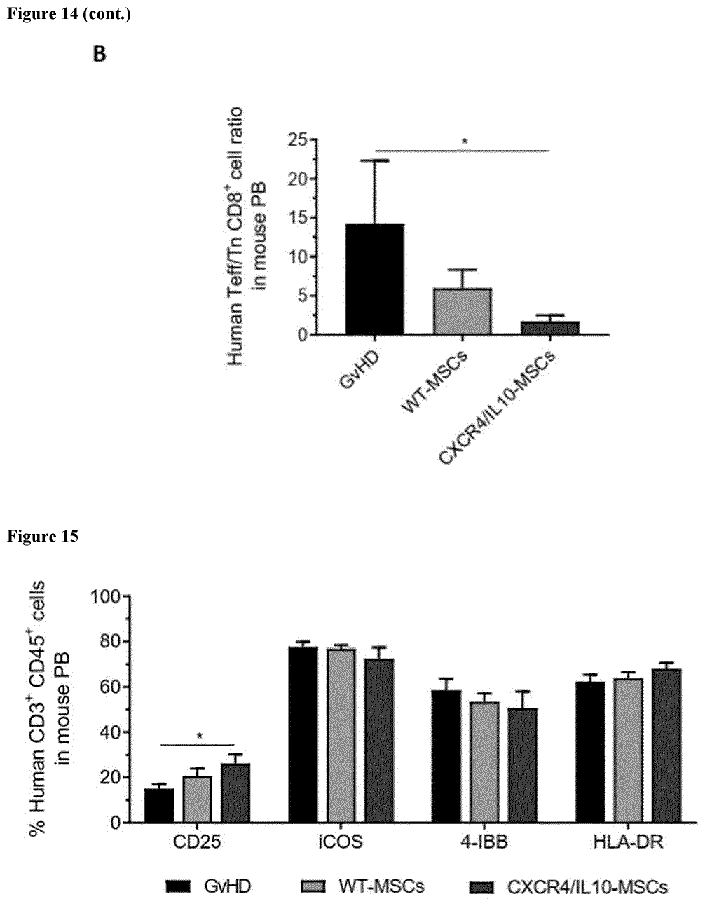

The analysis of the distribution of human CD4⁺ or CD8⁺ T cells in naïve, effector and memory T cells, showed a significant decrease in the percentage of CD4⁺ and CD8⁺T cells with effector phenotype in mice that received CXCR4/IL10-MSCs (FIGS. 14C and 14F).

The activation profile of circulating human T cells in the peripheral blood of mice was studied. The groups that received any type of Ad-MSCs showed an increase in the percentage of CD25⁺T cells, being statistically higher in mice treated with CXCR4/IL10-MSCs. Furthermore, these cells were CD25±CD4⁺ lymphocytes, which suggested the presence of circulating regulatory T cells in this group (FIG. 15).

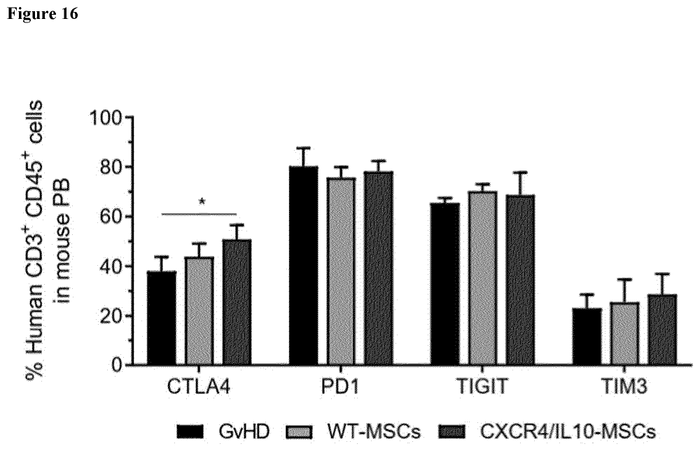

The exhaustion profile of circulating human CD3⁺T cells in peripheral blood was also analyzed using CTLA4, PD1, TIGIT and TIM3 markers. At three weeks post-transplantation of MNCs, an increase in circulating CD3⁺ cells positive for CTLA4 was observed in the two groups that received Ad-MSCS, being significantly higher in the case of the mice that received CXCR4/IL10-MSCs (FIG. 16).

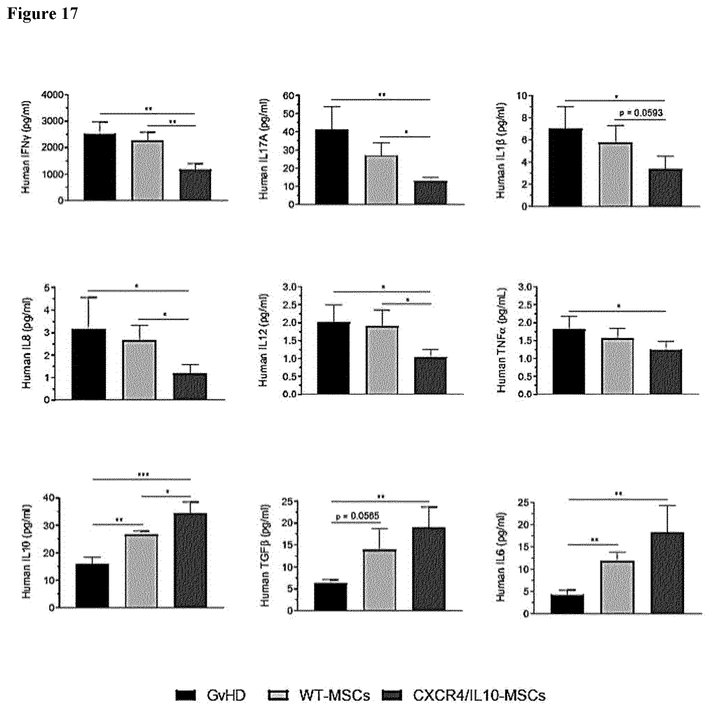

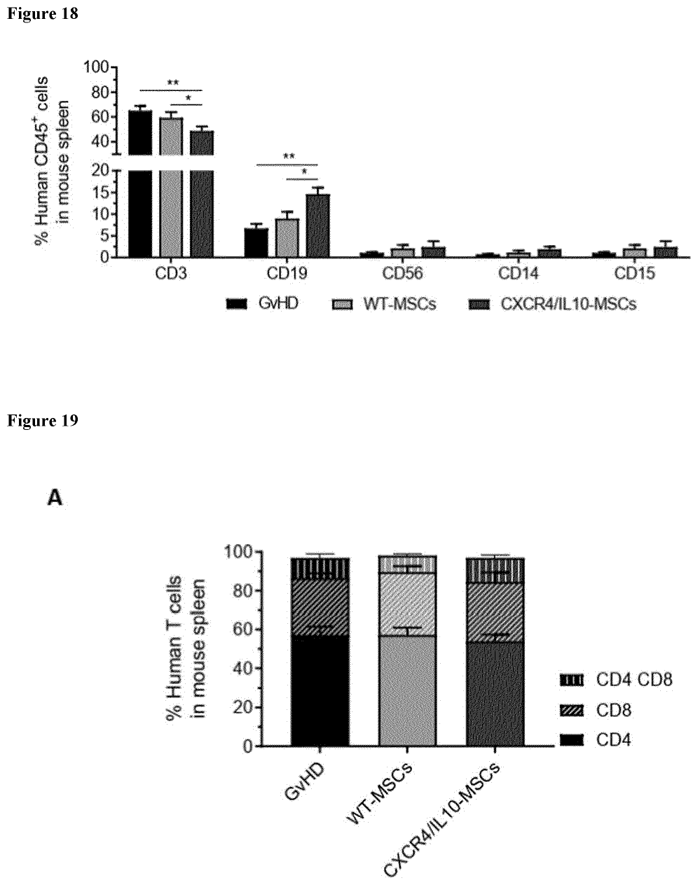

Circulating human cytokines and factors involved in the GvHD development were analyzed in the serum of these mice. As FIG. 17 shows, the groups treated with any type of Ad-MSCs presented a statistically significant decrease in the levels of circulating pro-inflammatory human cytokines such as IFNγ, IL17A, IL1α, IL8, IL12 or TNFα with respect to the GvHD control group. Additionally, these two groups that received Ad-MSCs experienced an increase in circulating human anti-inflammatory factors, such as IL10, TGF13 or IL6. Remarkably, changes in cytokine secretion from a pro-inflammatory to a more anti-inflammatory profile were statistically more marked in mice that received CXCR4/IL10-MSCs relative to those that received WT-MSCs (FIG. 17).

These results indicated that the infusion of CXCR4/IL10-MSCs produces a significant decrease in the percentage of circulating human T cells in peripheral blood with respect to values corresponding to mice treated with WT-MSCs. Additionally, peripheral blood T cells show a more immunosuppressive profile after infusion of CXCR4/IL10-MSCs compared to WT-MSCs.

Taken together, this data indicate that CXCR4/IL10-MSCs induce a significantly reduced inflammatory environment and enhanced immunoregulatory environment at the systemic level in NSG immunodeficient mice transplanted with human leukocytes.

The distribution among the different human hematopoietic lineages was studied in the spleen: CD3⁺T cells, CD19⁺B cells, CD56⁺NK cells, CD14⁺ monocytes and CD15+ granulocytes. About 70% of the human CD45⁺ cells observed in the spleen at three weeks post-transplantation in the GvHD group were human CD3⁺T cells (64.98±4.14%), while this percentage decreased in the group that received WT-MSCs (59.22±4.56%), and more markedly in the group that received CXCR4/IL10-MSCs (48.67±3.58%). Additionally, when the percentage of human CD19⁺B cells was analyzed in the spleen, a significant increase of this population was detected in the group treated with CXCR4/IL10-MSCs (14.62±1.52%) either compared to the GvHD group (6.73±1.03%) or to the group that received WT-MSCs (8.99±1.53%). Finally, the percentage of CD56⁺NK cells, CD14⁺ monocytes and CD15⁺ granulocytes in the spleen of transplanted mice was very low and without differences between the different study groups.

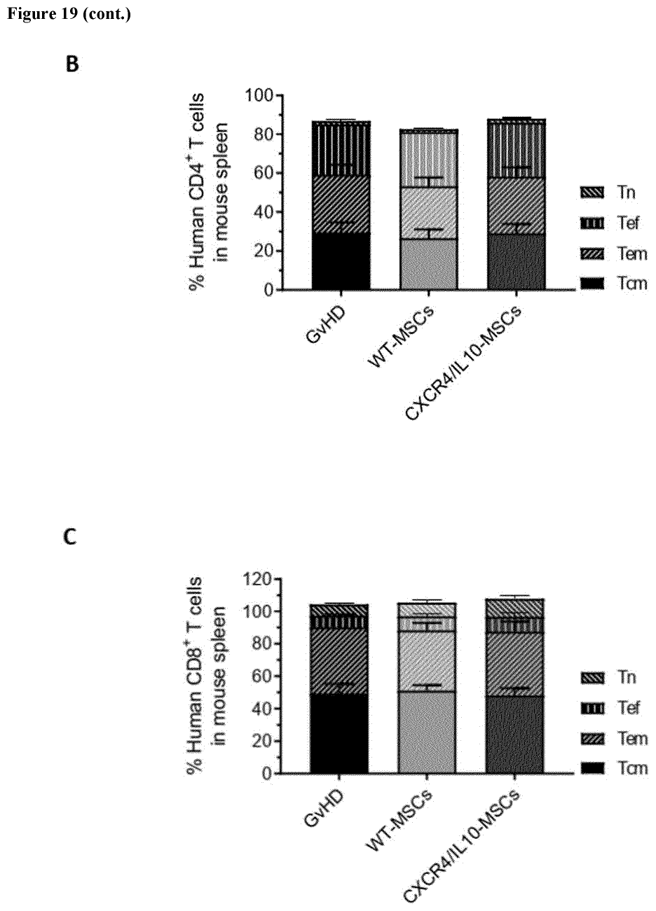

No significant differences were found in spleen between the study groups in the distribution of T cells between CD4⁺, CD8⁺ or double positive cells (FIG. 19A). No differences were observed between the different study groups in terms of the distribution of human CD4⁺ or CD8⁺T cells among the most characteristic subpopulations: naïve, effector and memory T cells (FIG. 19B and FIG. 19C).

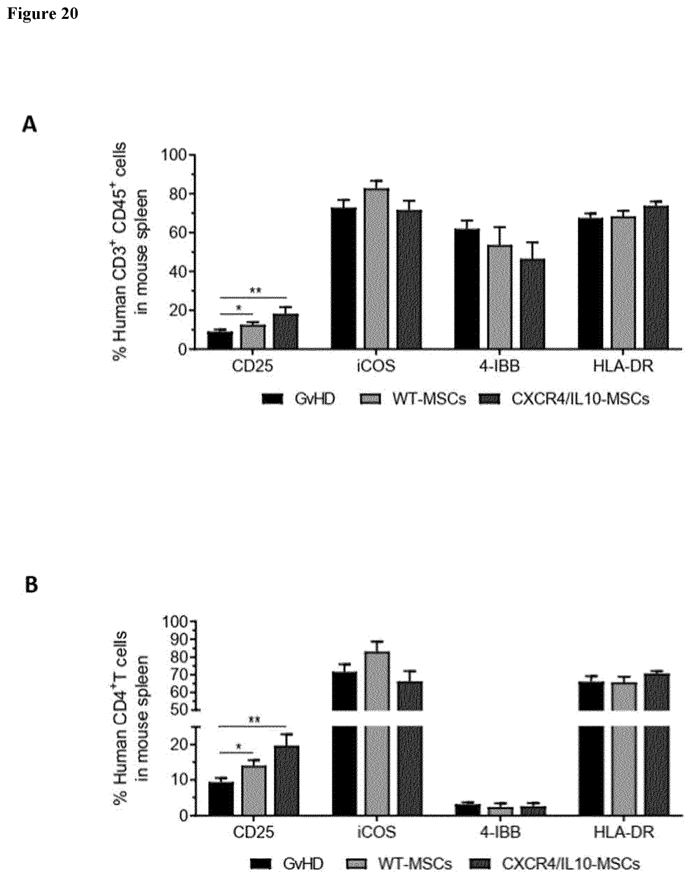

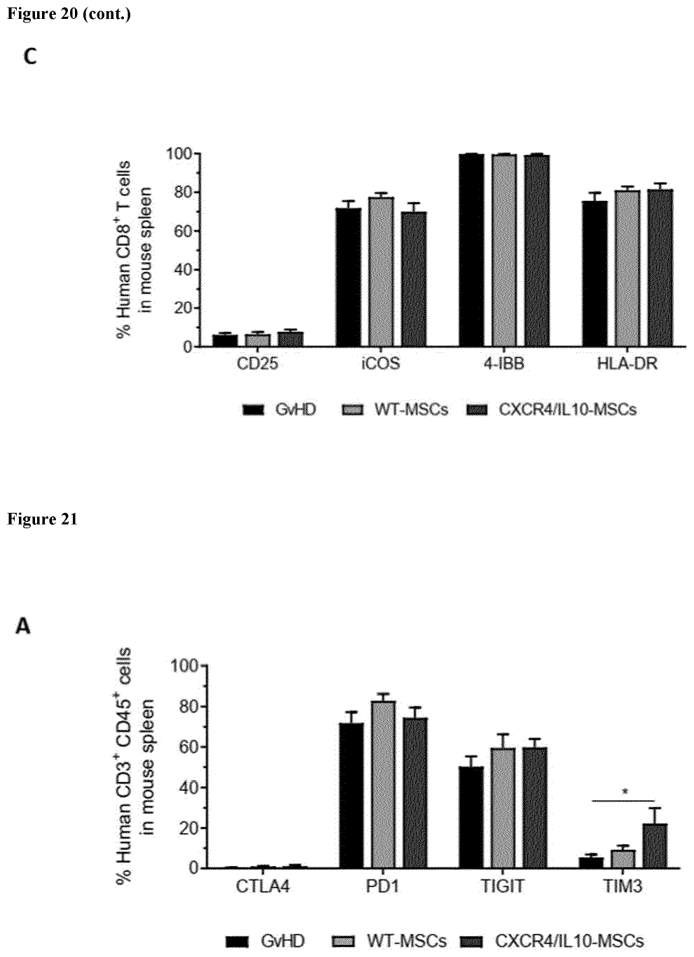

The activation pattern observed in the spleen was very similar to that observed in peripheral blood. Differences were only found between the groups in terms of the CD25 expression in the spleen (FIG. 20A). The groups that had received any type of Ad-MSCs showed a significant increase in the percentage of CD25⁺T cells with respect to the GvHD group, being higher in mice treated with CXCR4/IL10-MSCs compared to the group that received WT-MSCs. These cells specifically were CD25⁺CD4⁺T cells (FIG. 20B), indicating an immunoregulatory phenotype of these CD4⁺T cells in spleen.

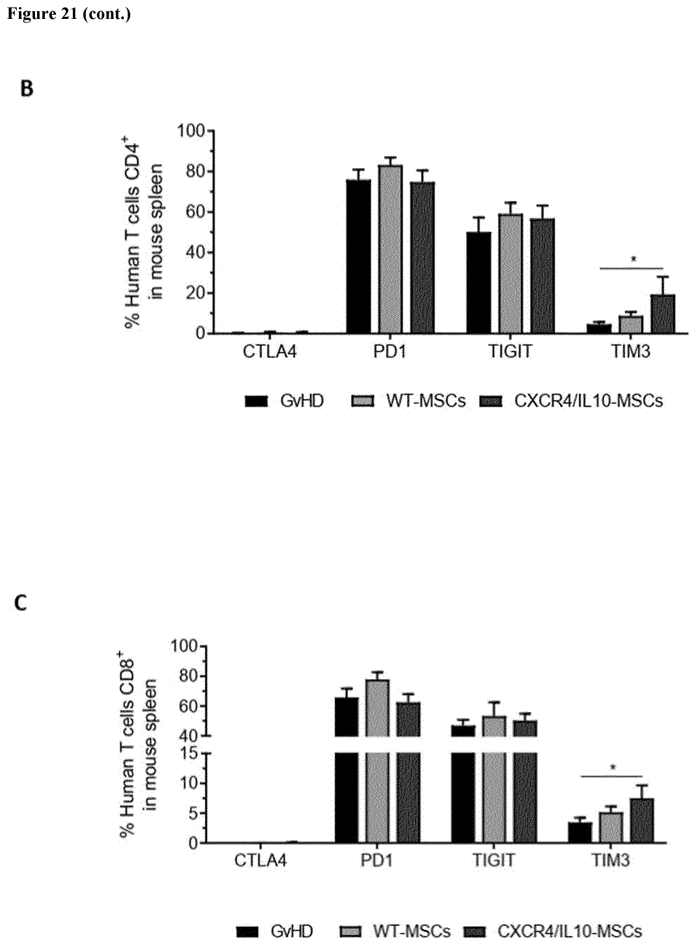

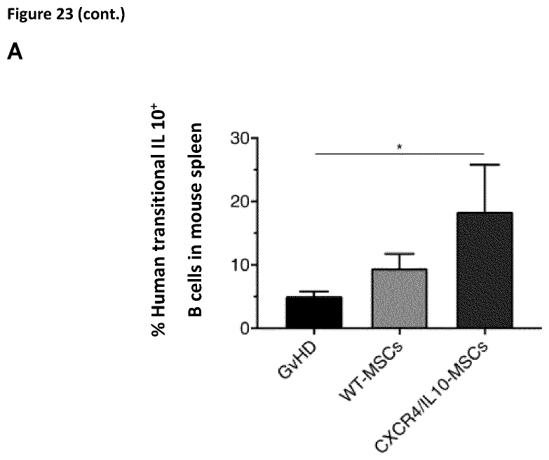

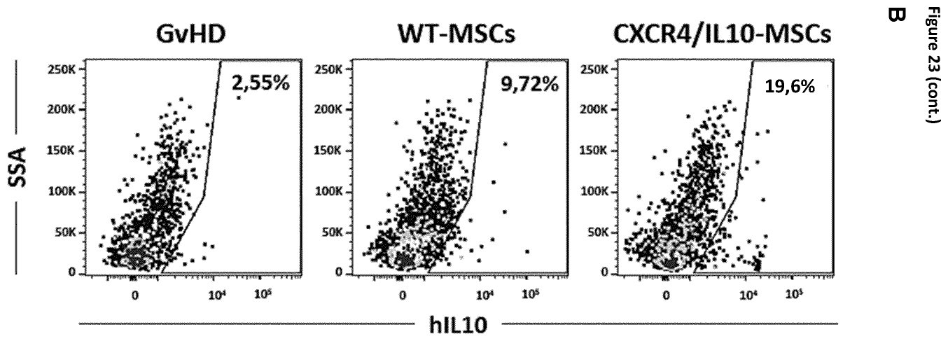

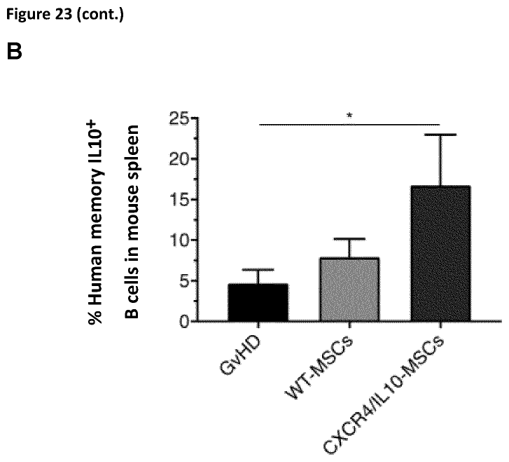

At three weeks post-transplantation of MNCs, the analysis of inhibition receptors in the spleen of NSG transplanted mice showed a significant increase in TIM3⁺T cells in human CD3⁺T cells (FIG. 23A) and also in CD4⁺ or CD8⁺ cells (FIGS. 21B and 21C, respectively) in mice treated with CXCR4/IL10-MSCs with respect to the GvHD control group of mice not treated with AdMSCs (FIG. 21). No differences were observed between the groups with respect to the other exhaustion markers analyzed.

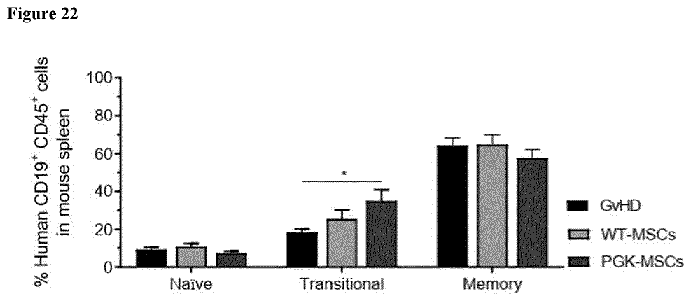

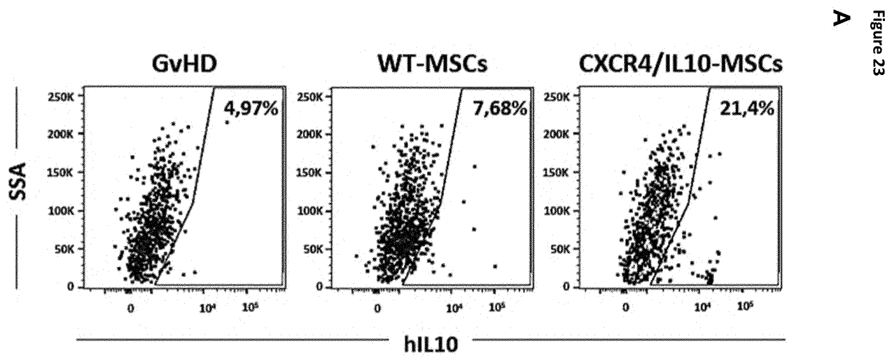

Flow cytometric analyses of B cells in the spleen showed that there was no change in the naïve B cell subpopulation between the non-MSC treated group and the groups receiving any type of AdMSCs. However, the percentage of transitional B cells, those that have not yet differentiated to antibody-producing B cells, was higher in mice that received CXCR4/IL10-MSCs (34.78±7.09%) compared to the group that received WT-MSCs (24.3±5.18%) and the GvHD control group not infused with any MSCs (17.47±2.21%). Finally, a slight decrease in the percentage of fully differentiated B cells was observed only in the group that received CXCR4/IL10-MSCs (FIG. 22).

These results suggested that WT-MSCs and more markedly CXCR4/IL10-MSCs were maintaining the B cell population in a transition state, without completing their differentiation into memory B cells or plasma cells

The percentage of Breg cells in the transitional B cell population was higher in mice that received WT-MSCs (FIG. 23A). Furthermore, this percentage was statistically higher in mice infused with CXCR4/IL10-MSCs. The same pattern was observed among the population of memory B cells secreting IL10 (FIG. 23B).

Taken together, these results suggest that the infusion of CXCR4/IL10-MSCs not only significantly favors the development of T cells with an immunoregulatory phenotype with respect to WT-MSCs, but is also improves the development of B cells with a beneficial effect against the development of GvHD.

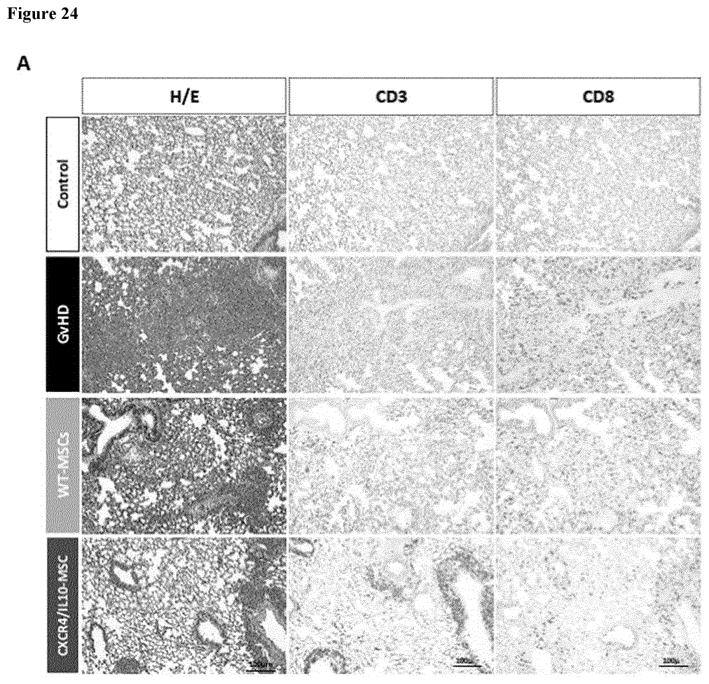

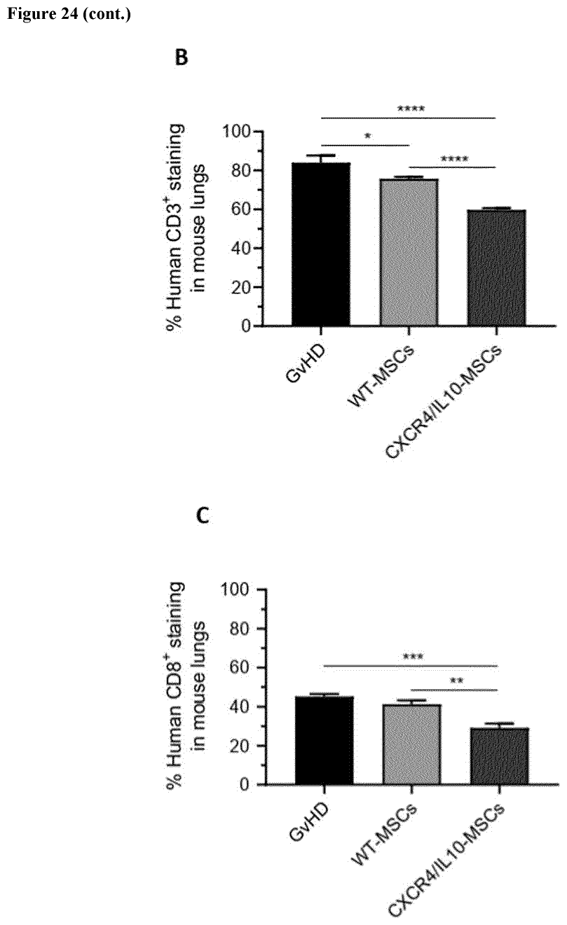

During the final phase of acute human GvHD, donor effector T cells mediate tissue injury in different organs through direct cytotoxic activity or the inflammatory cytokine production. Histopathological signs of GvHD were analyzed in target organs of this disease, such as the lungs or liver. Histological analysis of the lungs showed that mice that received CXCR4/IL10-MSCs presented much reduced infiltration of human T cells in the parenchyma with respect to the other two groups, which showed a structure similar to the control group without disease (FIG. 24A). By quantifying the presence of human CD3⁺ and CD8 T cells, it was found that the infusion of any type of Ad-MSCs reduced the percentage of both cell types in the lung. Remarkably, the reduction was much more significant, both for human CD3⁺ cells (FIG. 24B) and CD8⁺ cells (FIG. 24C), in mice treated with CXCR4/IL10-MSCs.

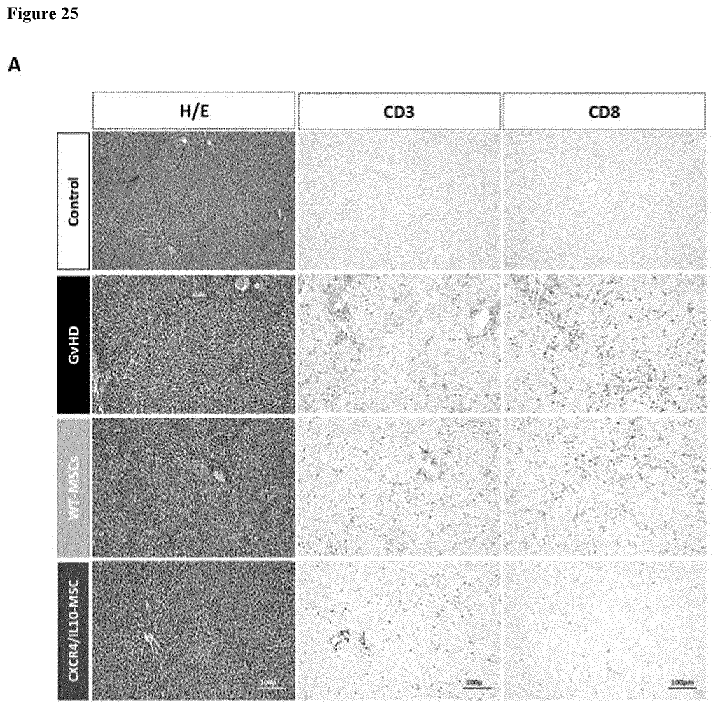

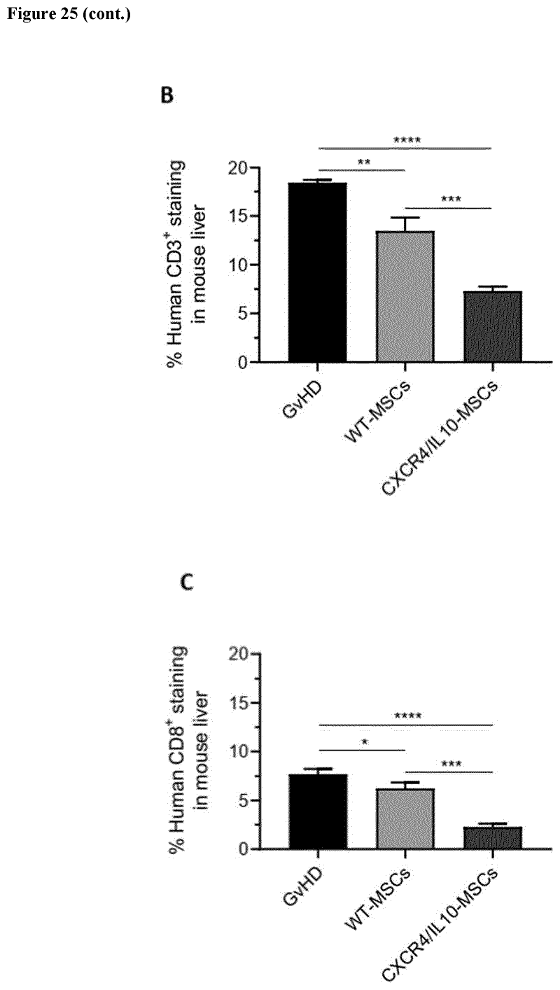

The histopathological analysis of the liver of transplanted mice showed human T cell infiltration levels of the parenchyma, and also perivascular inflammation, which were much reduced after the infusion of any type of Ad-MSCs. Even more, this inflammation was practically non-existent in mice treated with CXCR4/IL10-MSCs (FIG. 25A). On the other hand, the administration of Ad-MSCs significantly reduced the presence of human CD3⁺ and CD8⁺ cells in the liver, being this reduction much more significant when mice received CXCR4/IL10-MSCs (FIGS. 25B and 25C)

### Example 2.9. Enhanced Efficacy of CXCR4/1L10-MSCs Stably Expressing CXCR4 and IL10 in an Experimental Model of Inflammatory Bowel Disease (IBD) Induced by Dextran Sulphate (DSS)

We also tested the in vivo efficacy of genetically modified MSC expressing CXCR4 and IL10 in a new experimental model of inflammation: inflammatory bowel disease (IBD) induced by DSS.

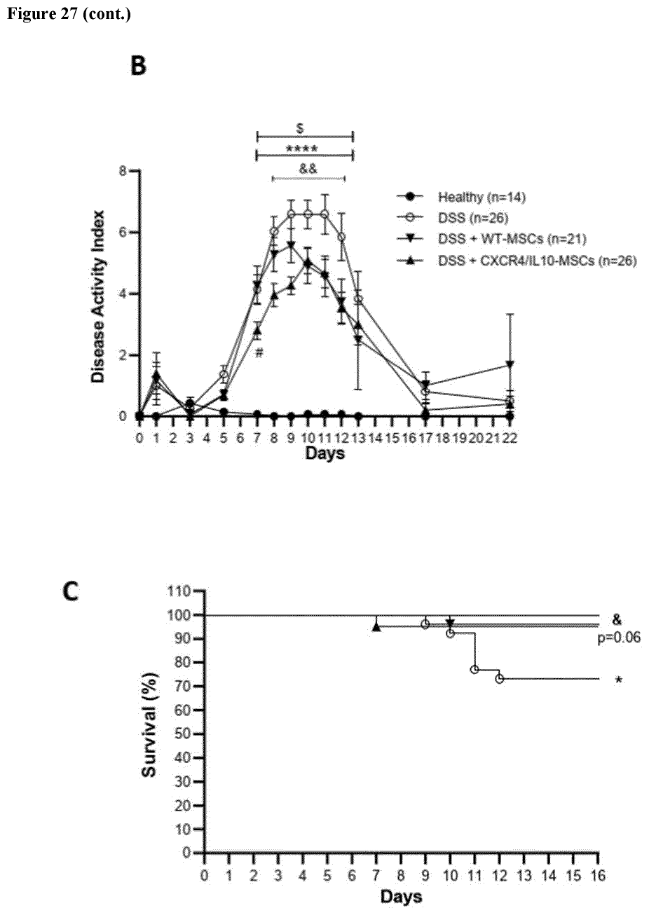

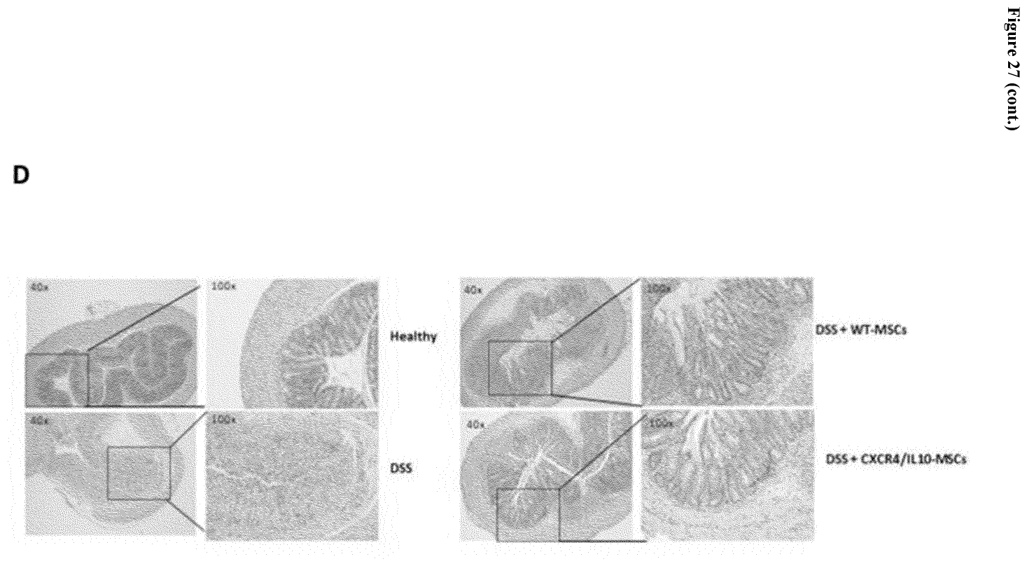

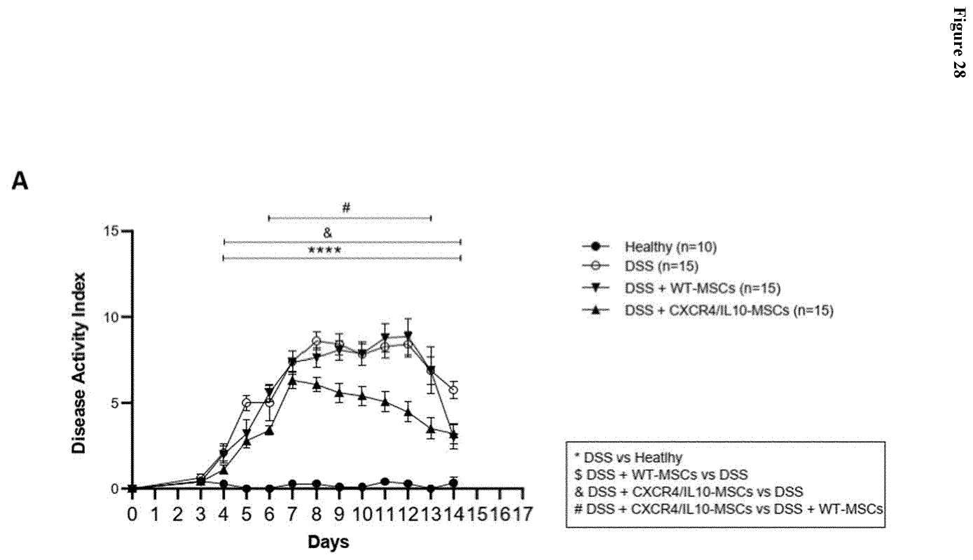

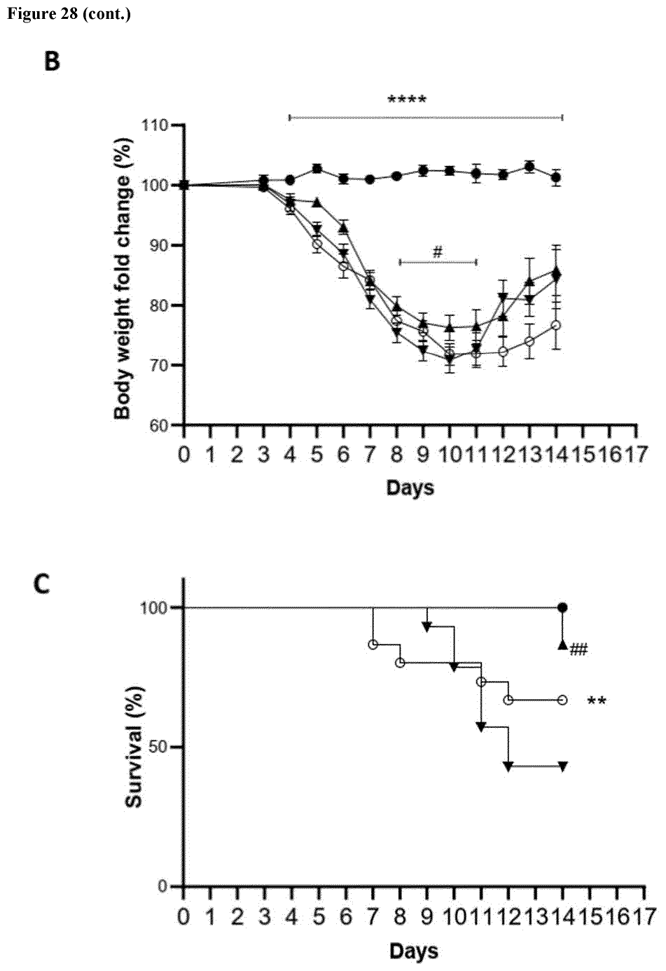

According to the experimental design shown in FIG. 26 (Materials and Methods), the disease activity index (DAI) in colitic mice treated with a single dose of CXCR4/IL10-MSCs was significantly lower either compared to mice not treated with MSCs or with mice treated with WT-MSCs (FIG. 27A). Also, significant differences were observed when the body weight loss (FIG. 27B) and the survival rate (FIG. 2FC) of CXCR4/IL10-MSCs treated mice were compared to the WT-MSC and the non-MSC treated groups during the first 7-day DSS cycle. Histologically, better preserved colon morphology and an attenuated leukocyte infiltration were observed in CXCR4/IL10-MSCs treated colitic mice, with respect to non-MSC-infused colitic mice (FIG. 27D) To study the long-term effects induced by CXCR4/IL10-MSCs in colitic mice, experiments were conducted according to the experimental design depicted in FIG. 26. A second challenge with a 7-day DSS cycle was conducted following a latency period of three months from the first DSS treatment. Infused CXCR4/IL10-MSCs induced a significantly reduced DAI (FIG. 28A), as well as less pronounced decrease in body weight (FIG. 28B) and survival (FIG. 28C), with respect to non-MSC-infused colitic mice.

These data show that CXCR4/IL10-MSCs have increased immunomodulatory properties compared to WT-MSCs in a DSS-induced model of colitis, indicating that these genetically-modified MSCs may represent a more potent MSC-based cell therapy product for the treatment of inflammatory bowel diseases, compared to WT MSCs.

1. A DNA expression cassette comprising:
a. A promoter;
b. A sequence encoding the chemokine receptor type 4 CXCR4; and
c. A sequence encoding interleukin IL-10.

2. The expression cassette, according to claim 1, characterized in that the sequence encoding the chemokine receptor type 4 CXCR4 is SEQ ID NO: 1 and the sequence encoding interleukin IL-10 is SEQ ID NO: 3.

3. The expression cassette, according to any of the claim 1 or 2, further comprising a regulatory element for increasing transgene expression.

4. The expression cassette, according to any of the claims 1 to 3, wherein the regulatory element is the woodchuck hepatitis virus regulatory element (WPRE) RNA export signal sequence or a functional variant or fragment thereof.

5. The expression cassette, according to any of the claims 1 to 4, further comprising, between the sequence encoding the chemokine receptor type 4 CXCR4 and the sequence encoding interleukin IL-10, a sequence which encodes an autocatalytic peptide.

6. The expression cassette, according to any of the claims 1 to 5, wherein the autocatalytic peptide is E2A, preferably SEQ ID NO: 2.

7. The expression cassette, according to any of the claims 1 to 6, wherein the promoter is a human phosphoglycerate kinase (PGK) promoter sequence or a functional homolog or variant thereof.

8. The expression cassette, according to any of the claims 1 to 7, comprising in the order 5′ to 3′:
a. A human phosphoglycerate kinase (PGK) promoter sequence or a functional homolog or variant thereof;
b. A sequence encoding the chemokine receptor type 4 CXCR4;
c. A sequence encoding the autocatalytic peptide E2A;
d. A sequence encoding interleukin IL-10; and
e. The woodchuck hepatitis virus post-transcriptional regulatory element (WPRE).

9. The expression cassette, according to any of the claims 1 to 8, comprising in the order 5 ‘ to 3’:
a. A human phosphoglycerate kinase (PGK) promoter sequence or a functional homolog or variant thereof;
b. SEQ ID NO: 1 encoding the chemokine receptor type 4 CXCR4;
c. SEQ ID NO: 2 encoding the autocatalytic peptide E2A;
d. SEQ ID NO: 3 encoding interleukin IL-10; and
e. The woodchuck hepatitis virus post-transcriptional regulatory element (WPRE).

10. A recombinant gene delivery integrative vector comprising the expression cassette of any of the claims 1 to 9.

11. The recombinant gene delivery integrative vector of claim 10 characterized in that it is a lentiviral vector.

12. A cell comprising the expression cassette of any of the claims 1 to 9, or the recombinant gene delivery integrative vector of any of claim 10 or 11.

13. The cell, according to claim 12, characterized in that it is a mesenchymal stem cell derived from bone marrow, placenta, umbilical cord, amniotic membrane, menstrual blood, peripheral blood, salivary gland, skin and foreskin, synovial fluid, amniotic fluid, endometrium, adipose tissue, cord blood and/or dental tissue.

14. A pharmaceutical composition comprising the recombinant gene delivery integrative vector of any of claim 10 or 11, or the cell of any of the claim 12 or 13 and, optionally, a pharmaceutically acceptable excipient or carrier.

15. Gene delivery integrative vector of any of claim 10 or 11, or the cell of any of the claim 12 or 13 for use as medicament.

16. Gene delivery integrative vector of any of claim 10 or 11, or the cell of any of the claim 12 or 13 for use, according to claim 15, in the treatment of inflammatory diseases.

17. Gene delivery integrative vector of any of claim 10 or 11 or the cell of any of the claim 12 or 13 for use, according to any of the claim 15 or 16, in the treatment of Graft-versus-host disease (GvHD), sepsis, rheumatoid arthritis, or inflammatory bowel disease.
